(19) United States
(12) Patent Application Publication
(10) Pub. No.: US 2014/0018406 A1
(43) Pub. Date: Jan. 16, 2014

(54) Treatment of Chronic Obstructive Pulmonary Disease With Nebulized Beta 2-Agonist or Combined Nebulized Beta 2-Agonist and Anticholinergic Administration

(71) Applicant: Sunovion Respiratory Development, Inc., Marlborough, MA (US)

(72) Inventors: William GERHART, La Mesa, CA (US); Ahmet TUTUNCU, San Diego, CA (US)

(73) Assignee: SUNOVION RESPIRATORY DEVELOPMENT, INC., Marlborough, MA (US)

(21) Appl. No.: 14/024,796

(22) Filed: Sep. 12, 2013

Related U.S. Application Data
(63) Continuation of application No. 12/797,537, filed on Jun. 9, 2010, now abandoned.
(60) Provisional application No. 61/185,524, filed on Jun. 9, 2009.
(60) Provisional application No. 61/185,528, filed on Jun. 9, 2009.

(51) Int. Cl. A61K 31/40; A61K 31/137

(52) U.S. Cl. CPC A61K 31/40; A61K 31/137
USPC 514/424

(57) ABSTRACT

Inhalation solutions for administration of beta 2-agonists or combinations of muscarinic antagonists and beta 2-agonists for the treatment of breathing disorders, such as COPD, are provided. The inhalation solutions are administered by nebulization, particularly with a high efficiency nebulizer.

This application is a continuation of U.S. patent application Ser. No. 12/797,537, filed Jun. 9, 2010, which claims priority under 35 U.S.C. §119(e) from U.S. Provisional Patent Application No. 61/185,524, filed Jun. 9, 2009, and from U.S. Provisional Patent Application No. 61/185,528, filed Jun. 9, 2009, each of which is incorporated herein by reference in its entirety.

### BACKGROUND OF THE INVENTION

Chronic obstructive airway disease (COPD) is a pulmonary (lung) disease characterized by chronic obstruction of the airways. COPD encompasses emphysema and chronic bronchitis. Chronic bronchitis is diagnosed where a patient suffers from chronic cough, mucus production, or both, for at least three months in at least two successive years where other causes of chronic cough have been excluded. In chronic bronchitis, airway obstruction is caused by chronic and excessive secretion of abnormal airway mucus, inflammation, and bronchospasm. Often chronic bronchitis is exacerbated by frequent or chronic infection.

Emphysema involves the destruction of elastin in terminal bronchioles, which leads to remodeling, destruction and ultimate collapse of the airway walls. Patients with emphysema gradually lose the ability to exhale, causing a rise in blood waste gasses (such as carbon dioxide), a drop in blood oxygen, and a general degradation of patient stamina and overall health. A characteristic of emphysema is permanent loss of alveoli. Remodeling leads to permanent enlargement of the air spaces distal to the terminal bronchioles, and destruction of terminal bronchiole walls, though without fibrosis. Emphysema is progressive with a poor prognosis. Since there is no known method for repairing elastin or restoring the alveoli, therapy is generally palliative and persistent.

Most patients suffering from COPD have both emphysema and chronic bronchitis. The standard of treatment for COPD includes maintenance and/or rescue dosing of bronchodilator and/or anti-inflammatory aerosol drugs. While most patients respond to treatment with metered dose inhalers or dry powder inhalers, there is a subset of patients for whom such options are not well-suited. Older and sicker COPD patients, for example, often find it difficult to use, or do not experience therapeutic benefit from the use of, metered dose inhalers or dry powder inhalers.

Dry powder inhalers are generally passive delivery devices, which patients actuate by forceful, controlled inhalation through the mouth. Metered dose inhalers, on the other hand, are in general active delivery devices, which create an atomized mist by forcing a drug solution or suspension through a nozzle under pressure. A patient activates the metered dose inhaler by pressing an actuator and simultaneously breathing in through the mouth in order to deposit the drug in the patient's lungs. Patients whose motor skills are impaired or not fully developed will often have trouble activating the device, coordinating their breathing, and generally using metered dose inhalers. Patients who also have poor inhalation capacity and control find dry powder inhalers to be difficult to operate as well. Newer inhaler devices that are breath-actuated or produce a soft mist are easier for patients to operate; but these newer devices still require coordination and a breath-hold; and achievement of sufficient lung deposition and distribution is reliant on only one or two inhalations. For sicker and older COPD patients, nebulizer delivery of their medicines is an important delivery option, since they can generally receive a full dose regardless of disease state, because all that is required is normal (tidal) breathing over multiple minutes.

There are two general categories of bronchodilators effective for treating COPD—muscarinic antagonists and beta 2-agonists. Longer-acting bronchodilators are preferred to shorter-acting bronchodilators due to their superior efficacy and duration of effect, as well as favorable impact on patient compliance.

Three FDA approved long-acting beta 2-agonists (so called LABAs) that have been approved for use in COPD in the United States are formoterol fumarate (Foradil®, Perforomist®), arformoterol tartrate (Brovana®), and salmeterol xinafoate (Serevent®). Each of these LABAs have only been approved for twice-daily dosing, having demonstrated clinically meaningful bronchodilation with acceptable side effects over only 12 hours. One LAB A, arformoterol tartrate, demonstrated clinically meaningful bronchodilation over 24 hours in a clinical trial, but with unacceptable side effects. R. Baumgartner, et al., “Nebulized Arformoterol in Patients with COPD: A 12-Week, Multicenter, Randomized, Double-Blind, Double-Dummy, Placebo- and Active-Controlled Trial,” Clinical Therapeutics, Vol. 29, No. 2, 2007.

One long-acting muscarinic antagonist (so called LAMA) that has been approved for use in COPD in the United States is tiotropium bromide powder for inhalation (Spiriva®, NDA No. 021395, Boehringer Ingelheim). Tiotropium bromide is available commercially only as a dry powder, which is administered by a breath-activated inhaler. A similar mode of administration is disclosed by Bannister et al. (U.S. Pat. No. 7,229,607) for administration of glycopyrronium bromide (glycopyrrolate) as a dry powder. The '607 patent claims a method for achieving grater than 20 hours of bronchodilation in a COPD patient by means of coated particles in a dry powder formulation. The '607 patent distinguishes this methodology from administration of a solution formulation of glycopyrrolate, which is characterized as being unable to achieve effective treatment of COPD for longer than 12 hours. For example, Bannister et al. state: “Schroeckenstein et al., J. Allergy Clin. Immunol, 1988; 82(1): 115-119, discloses the use of glycopyrrolate in an aerosol formulation for treating asthma. A single administration of the metered-dose glycopyrrolate aerosol achieved bronchodilation over a 12 hour period.” Additionally, Bannister et al. admit: “Skorodin, Arch Intern. Med, 1993; 153: 814 828, discloses the use of glycopyrrolate in an aerosol formulation for the treatment of asthma and COPD. It is stated that, in general, the quaternary ammonium anticholinergic compounds have a duration of action of 4 to 12 hours. A dose of between 0.2 to 1.0 mg of glycopyrrolate is recommended at 6 to 12 hour intervals.” And the inventors of the '607 patent also state: “Walker et al., Chest, 1987; 91(1): 49-51, also discloses the effect of inhaled glycopyrrolate as an asthma treatment. Again, the duration of effective treatment is shown to be up to 12 hours, although up to 8 hours appears to be maximal.”

The combination of a LABA and a LAMA may offer synergistic benefits. As of yet, no LABA/LAMA combinations have been approved by any regulatory authority, although several are in development. There have been numerous fixed combinations consisting of two active pharmaceutical ingredients developed and approved for COPD (e.g. Advair®, Combivent®, DuoNeb®), but in every case the dose, and the frequency of dosing, approved was the same as that for the individual active pharmaceutical ingredient monotherapies.

A sub-segment of the COPD population comprising the sickest and oldest patients requires nebulizer delivery of their medicines because they are unable to satisfactorily operate a metered dose or dry powder inhaler, or because they experience superior therapeutic benefit from nebulizer delivery of the medications. However, the treatment options for these patients are limited. Two long-acting beta 2 agonist solution formulations are approved for nebulizer delivery twice daily (B.I.D.), and indicated for the maintenance treatment of COPD symptoms. However, once-daily (Q.D.) dosing is preferable to B.I.D. There are no LAMAs approved for nebulizer delivery. Ipratropium bromide is the only muscarinic antagonist approved for nebulizer delivery in COPD (monotherapy or in combination with albuterol), however ipratropium +/−albuterol is indicated for administration four times per day (QID); and QID dosing and long nebulization times of this short-acting agent is inconvenient, leading to poor compliance and thus sub-optimal clinical outcomes. Glycopyrrolate has been demonstrated to potentially be a safe and effective bronchodilator that provides up to 12 hours of clinically meaningful improvement in therapeutic bronchodilation in COPD patients with acceptable side effects when delivered by a nebulizer. Longer acting aerosol drugs have been demonstrated to generally be more efficacious and result in better compliance compared to shorter acting drugs. Furthermore, it has not been previously demonstrated that combining a LABA, previously demonstrated to provide only 12 hours of clinically meaningful duration of bronchodilation with acceptable side effects, with a LAMA, that previously demonstrated only up to 12 hours of clinically meaningful bronchodilation with acceptable side effects in a nebulizer, can result in 24 hours of clinically meaningful bronchodilation with acceptable side effects or a significantly improved therapeutic index.

There is thus a need for additional therapeutic options for the treatment of COPD. There is a need for therapeutic options that offer greater convenience, better efficacy, and/or better safety, especially for the sub-population of COPD patients who require nebulizer delivery. In particular there is a need for a nebulized beta 2-agonist that provides more than 12 hours, and preferably at least 24 hours of therapeutic benefit to COPD patients. There is also a need for a fixed combination of a LABA/LAMA that provides 24 hours of therapeutic benefit to COPD patients wherein the LABA and/or the LABA previously have been demonstrated to provide only 12 hours of clinically meaningful therapeutic benefit with acceptable side effects. And, there is a need for a fixed combination of a LABA/LAMA wherein, although no improvement in duration of therapeutic benefit may be seen compared to the individual active monotherapies, a significant improvement is provided in the safety profile. Heretofore, no methods, devices or systems have been suggested that satisfies these needs.

There is a need for more effective approaches to treating COPD.

### SUMMARY OF THE INVENTION

The invention provides methods of treating COPD and a device or system adapted for such treatment. In particular, the invention provides methods and systems for treating COPD by administering a long-acting beta 2-agonist (LABA) or a combination of a long-acting muscarinic antagonist (LAMA) and a LABA to a patient in need of such treatment. Embodiments described herein provide improved therapeutic efficacy (e.g. enhanced duration and/or magnitude of therapeutic effect), improvements in the side effects generally associated with LAMA and/or LABA therapy, and/or improved patient compliance (e.g. due to improved convenience, reduced side effects, improved overall feeling of wellness, etc.).

Provided herein is a method of treating a patient having chronic obstructive pulmonary disease (COPD), comprising administering to the patient, with a high efficiency nebulizer, a dose of a long-acting beta 2-agonist (LABA) that produces a significantly improved therapeutic effect in the patient compared to administration of the same dose of the LABA with a conventional nebulizer, metered dose inhaler or dry powder inhaler. In some embodiments, administering the LABA with the high efficiency nebulizer results in significantly improved magnitude or duration of therapeutic effect, and/or significantly improved side effects, compared to administering the LABA with a conventional nebulizer, a metered dose inhaler, or a dry powder inhaler. In some embodiments, the dose of the LABA is an amount of the LABA that produces clinically meaningful bronchodilation for at least 24 hours when administered with a high efficiency nebulizer, wherein the same LABA produces significantly less than 24 hours clinically meaningful bronchodilation when administered with a conventional nebulizer, a metered dose inhaler or a dry powder inhaler. In some embodiments, the clinically meaningful bronchodilation is an increase in trough FEV₁ of at least 10% or at least 100 mL above placebo. In some embodiments, the dose of the LABA is an amount of the LABA that produces clinically meaningful bronchodilation for at least 24 hours, with acceptable side effects, when administered with a high efficiency nebulizer, and wherein a dose of the same LABA produces significantly less than 24 hours of clinically meaningful bronchodilation, with acceptable side effects, when administered to the lungs with a conventional nebulizer, a metered dose inhaler or a dry powder inhaler. In some embodiments, the LABA that is administered comprises formoterol, salmeterol, indacaterol, or a pharmaceutically acceptable enantiomer and/or salt thereof.

Also provided herein is a method of treating a patient having chronic obstructive pulmonary disease (COPD), comprising administering to the patient a LABA, with a high efficiency nebulizer, wherein such administration significantly improves the duration and/or magnitude of therapeutic effect of the LABA, while retaining acceptable side effects, compared to administering the same LABA administered with a conventional nebulizer, metered dose inhaler or dry powder inhaler. In some embodiments, administering the LABA with the high efficiency nebulizer results in clinically meaningful bronchodilation for at least 24 hours, with acceptable side effects, and wherein administering the same LABA with a conventional nebulizer, metered dose inhaler or dry powder inhaler results in significantly less than 24 hours of clinically meaningful bronchodilation with acceptable side effects. In some embodiments, the LABA is formoterol, salmeterol, or a pharmaceutically acceptable enantiomer and/or salt thereof.

Also provided herein is a method of treating a patient having chronic obstructive pulmonary disease (COPD), comprising administering to the patient with a high efficiency nebulizer a reduced dose of a long-acting beta 2-agonist (LABA), wherein said reduced dose of LABA is less than half of an approved therapeutic dose of LABA administered with a conventional nebulizer, a metered dose inhaler, or a dry powder inhaler and wherein the reduced dose of LABA provides (a) similar magnitude of therapeutic effect; (b) similar duration of therapeutic effect; or both (a) and (b), compared with administration of the approved therapeutic dose of LABA with a conventional nebulizer, a metered dose inhaler, or a dry powder inhaler. In some embodiments, the LABA is formoterol, salmeterol, indacaterol, or a pharmaceutically acceptable enantiomer and/or salt thereof. In some embodiments, administration of the LABA with the high efficiency nebulizer results in reduced side effects compared to the approved therapeutic dose of the LABA administered with a conventional nebulizer, a metered dose inhaler, or a dry powder inhaler. In some embodiments, the LABA is formoterol, or a pharmaceutically acceptable salt thereof, and is administered at a dose of less than about 10 μg. In some embodiments, the LABA is R,R-formoterol, or a pharmaceutically acceptable salt thereof, and is administered at a dose of less than about 7.5 μg of enantiomerically pure R,R-formoterol. In some embodiments, the LABA is salmeterol, or a pharmaceutically acceptable salt thereof, and is administered at a dose of less than about 25 μg.

Also provided is a method of treating a patient having chronic obstructive pulmonary disease (COPD), comprising administering to the patient with a high efficiency nebulizer a dose of a long-acting beta 2-agonist (LABA), wherein said administration provides: (i) an increased magnitude of therapeutic effect; (ii) an increased duration of therapeutic effect; and/or (iii) reduced side effects, as compared to administration of a dose of the LABA, with a conventional nebulizer, sufficient to achieve the same respirable or deposited dose as is achieved with the high efficiency nebulizer. In some embodiments, the LABA is formoterol, salmeterol, indacaterol, or a pharmaceutically acceptable enantiomer and/or salt thereof.

Also described herein is a method of treating a patient having chronic obstructive pulmonary disease (COPD), comprising administering to the patient with a high efficiency nebulizer a dose of long-acting beta 2-agonist (LABA), wherein said administration provides substantially the same magnitude and duration of therapeutic effect, and reduced side effects, as compared to administration of a dose of the LABA, with a conventional nebulizer, metered dose inhaler or dry powder inhaler that is necessary to achieve the same respirable or deposited dose as is achieved with the high efficiency nebulizer. In some embodiments, the LABA is formoterol, salmeterol, indacaterol, or a pharmaceutically acceptable enantiomer and/or salt thereof.

Also provided herein is a method of treating a patient having chronic obstructive pulmonary disease (COPD), comprising administering to the patient, with a high efficiency nebulizer, a dose of a combination of an amount of a long-acting beta 2-agonist (LABA) and an amount of a long-acting muscarinic antagonist (LAMA), wherein administering the dose of the combination with the high efficiency nebulizer is effective to produce a significantly improved therapeutic effect in the patient compared to administration of the LABA with a nebulizer as a monotherapy, and/or compared to administration of the LAMA with a nebulizer as a monotherapy. In some embodiments, administering the dose of the combination with the high efficiency nebulizer results in significantly improved magnitude or duration of therapeutic effect, and/or significantly improved side effects, compared to administering the LABA with a nebulizer as a monotherapy and/or compared to administering the LAMA with a nebulizer as a monotherapy. In some embodiments, the dose of the combination refers to the nominal, respirable or deposited dose of the combination. In some embodiments, the dose of the combination is an amount of the LABA that produces clinically meaningful bronchodilation for significantly less than 24 hours, with acceptable side effects, when administered with a nebulizer and/or an amount of the LAMA that produces clinically meaningful bronchodilation for significantly less than 24 hours, with acceptable side effects, when administered with a nebulizer, wherein the dose of the combination produces clinically meaningful bronchodilation for at least 24 hours, with acceptable side effects, of when administered with a high efficiency nebulizer. In some embodiments, administering the dose of the combination with the high efficiency nebulizer is effective to produce a significantly improved therapeutic effect in the patient compared to administering the LABA with a conventional nebulizer as a monotherapy, and/or compared to administering the LAMA with a conventional nebulizer as a monotherapy. In some embodiments, the clinically meaningful bronchodilation is an increase in trough FEV₁ of at least 10% or 100 mL above placebo. In some embodiments, the LABA is formoterol, salmeterol, indacaterol, or a pharmaceutically acceptable enantiomer and/or salt thereof. In some embodiments, the LAMA is glycopyrrolate or a pharmaceutically acceptable enantiomer and/or salt thereof. In some embodiments, the LABA is formoterol or a pharmaceutically acceptable enantiomer and/or salt thereof and the LAMA is glycopyrrolate or a pharmaceutically acceptable enantiomer and/or salt thereof.

Also provided is a method of treating a patient having chronic obstructive pulmonary disease (COPD), comprising administering to the patient, with a high efficiency nebulizer, a dose of a combination of an amount of a long-acting beta 2-agonist (LABA) and an amount of a long-acting muscarinic antagonist (LAMA), wherein administering the dose of the combination with the high efficiency nebulizer is effective to produce a significantly improved therapeutic effect in the patient compared to administration of the LABA with a nebulizer, metered dose inhaler, or dry powder inhaler as a monotherapy, and/or compared to administration of the LAMA with a nebulizer, soft mist inhaler, metered dose inhaler, or dry powder inhaler as a monotherapy. In some embodiments, administering the dose of the combination with the high efficiency nebulizer results in significantly improved magnitude or duration of therapeutic effect, and/or significantly improved side effects, compared to administering the LABA with a nebulizer, metered dose inhaler, or dry powder inhaler as a monotherapy and compared to administering the LAMA with a nebulizer as a monotherapy. In some embodiments, the dose of the combination refers to the nominal, respirable or deposited dose of the combination. In some embodiments, the dose of the combination is an amount of the LABA that produces clinically meaningful bronchodilation with acceptable side effects for significantly less than 24 hours when administered with a nebulizer, metered dose inhaler, or dry powder inhaler and/or an amount of the LAMA that produces clinically meaningful bronchodilation with acceptable side effects for significantly less than 24 hours when administered with a nebulizer, soft mist inhaler, metered dose inhaler, or dry powder inhaler, wherein the dose of the combination produces clinically meaningful bronchodilation with acceptable side effects for at least 24 hours when administered with a high efficiency nebulizer. In some embodiments, administering the dose of the combination with the high efficiency nebulizer is effective to produce a significantly improved therapeutic effect in the patient compared to administration of the LABA with a conventional nebulizer as a monotherapy, and/or compared to administration of the LAMA with a conventional nebulizer as a monotherapy. In some embodiments, the clinically meaningful bronchodilation is an increase in trough FEV₁ of at least 10% or 100 mL above placebo. In some embodiments, the LABA is formoterol, salmeterol, indacaterol, or a pharmaceutically acceptable enantiomer and/or salt thereof. In some embodiments, the LAMA is glycopyrrolate or a pharmaceutically acceptable enantiomer and/or salt thereof. In some embodiments, the LABA is formoterol, salmeterol, indacaterol, or a pharmaceutically acceptable enantiomer and/or salt thereof and the LAMA is glycopyrrolate or a pharmaceutically acceptable enantiomer and/or salt thereof.

Also provided herein is a method of treating a patient having chronic obstructive pulmonary disease (COPD), comprising twice per day administering to the patient, with a high efficiency nebulizer, a dose of a combination of an amount of a long-acting beta 2-agonist (LABA) and an amount of a long-acting muscarinic antagonist (LAMA), wherein administering the dose of the combination twice per day with the high efficiency nebulizer is effective to elicit significantly reduced side effects in the patient compared to twice per day administration of the LABA with a nebulizer as a monotherapy, and/or compared to twice per day administration of the LAMA with a nebulizer as a monotherapy. In some embodiments, the amount of the LABA in the combination dose is significantly reduced compared to a twice per day dose of the LABA as a monotherapy. In some embodiments, the amount of the LAMA in the combination dose is significantly reduced compared to a twice per day dose of the LAMA as monotherapy.

Some embodiments described herein provide a method of treating a patient having chronic obstructive pulmonary disease (COPD), comprising administering to the patient an amount of formoterol or a combination of glycopyrrolate and formoterol sufficient to produce a therapeutic effect for at least 24 hours with acceptable side effects.

Some embodiments described herein provide a method of treating a patient having chronic obstructive pulmonary disease (COPD), comprising administering to the patient with a high efficiency nebulizer a nominal, respirable, or deposited dose of formoterol, wherein said administration provides: (i) an increased magnitude of therapeutic effect; (ii) an increased duration of therapeutic effect; and/or (iii) reduced side effects, as compared to administration of the same nominal, respirable, or deposited dose of formoterol with a conventional nebulizer. Some embodiments described herein provide a method of treating a patient having chronic obstructive pulmonary disease (COPD), comprising administering to the patient with a high efficiency nebulizer a nominal dose of formoterol, wherein said administration provides: an increased magnitude and/or duration of therapeutic effect and therapeutically acceptable side effects, as compared to administration of the same nominal dose of formoterol with a conventional nebulizer. Some embodiments described herein provide a method of treating a patient having chronic obstructive pulmonary disease (COPD), comprising administering to the patient with a high efficiency nebulizer a respirable or deposited dose of formoterol, wherein said administration provides: (i) a similar magnitude and/or duration of therapeutic effect; and reduced side effects, as compared to administration of the same respirable or deposited dose of formoterol with a conventional nebulizer.

Some embodiments described herein provide a method of treating a patient having chronic obstructive pulmonary disease (COPD), comprising administering to the patient with a high efficiency nebulizer an amount of a LABA, e.g. formoterol, effective to provide a therapeutic effect, with acceptable side effects, for at least 24 hours.

Some embodiments described herein provide a method of treating a patient having a respiratory condition, comprising administering to the patient with a high efficiency nebulizer a nominal, respirable, or deposited dose of a LABA, wherein said administration provides: (i) an increased magnitude of therapeutic effect; (ii) an increased duration of therapeutic effect; and/or (iii) reduced side effects, as compared to administration of the same nominal, respirable, or deposited dose of said LABA with a conventional nebulizer.

### INCORPORATION BY REFERENCE

Any and all references cited herein are incorporated herein by reference in their entirety.

### DETAILED DESCRIPTION OF THE INVENTION

Unless defined otherwise, all technical and scientific terms used herein have the same meanings as are commonly understood by one of skill in the art to which the inventions described herein belong. All publications, patents, and patent applications mentioned in this specification are herein incorporated by reference to the same extent as if each individual publication, patent, or patent application was specifically and individually indicated to be incorporated by reference.

### DEFINITION OF TERMS

As used herein, the term “about” is used synonymously with the term “approximately.” Illustratively, the use of the term “about” with regard to a certain therapeutically effective pharmaceutical dose indicates that values slightly outside the cited values, e.g., plus or minus 0.1% to 10%, which are also effective and safe.

As used herein, the terms “comprising”, “including”, “such as”, and “for example” (or “e.g.”) are used in their open, non-limiting sense.

As used herein “meg” means micrograms, and is synonymous with “μg” or “ug”. One microgram (mcg) is 0.001 mg, or 0.000001 g.

As used herein, the phrase “consisting essentially of” is a transitional phrase used in a claim to indicate that the following list of ingredients, parts or process steps must be present in the claimed composition, machine or process, but that the claim is open to unlisted ingredients, parts or process steps that do not materially affect the basic and novel properties of the invention.

“Nominal dose”, as used herein, refers to the loaded dose, which is the amount of active pharmaceutical ingredient (“API”) in an inhalation device prior to administration to the patient. The volume of solution containing the nominal dose is referred to as the “fill volume”.

“AUC₍₀₋ₜ₎^{HEN}” as used herein, refers to the area under a blood plasma concentration curve up to the last time point for the nominal dose of active pharmaceutical ingredient (API) administered with a high efficiency nebulizer.

“AUC₍₀₋ₜ₎^{Conv}” as used herein, refers to the area under a blood plasma concentration curve up to the last time point for a nominal dose of active pharmaceutical ingredient (API) administered with a conventional nebulizer.

“AUC_{(0-∞)}^{HEN}” as used herein, refers to the area under a blood plasma concentration curve for a nominal dose of active pharmaceutical ingredient (API) administered with a high efficiency nebulizer.

“AUC_{(0-∞)}^{Conv}” as used herein, refers to the area under a blood plasma concentration curve for a nominal dose of active pharmaceutical ingredient (API) administered with a conventional nebulizer [AUC_{(0-∞)}^{Conv}].

“Substantially the same nominal dose” as used herein, means that a first nominal dose of an active pharmaceutical ingredient (API) contains approximately the same number of millimoles of the muscarinic antagonist as a second nominal dose of the muscarinic antagonist.

“Substantially the same nominal dose” as used herein, means that a first nominal dose of an active pharmaceutical ingredient (API) contains approximately the same number of millimoles of the muscarinic antagonist as a second nominal dose of the muscarinic antagonist.

“Bioavailability” as used herein, refers to the amount of unchanged drug that reaches the systemic circulation. By definition, the bioavailability of an intravenous solution containing the active pharmaceutical ingredient (API) is 100%.

“Enhanced lung deposition,” as used herein, refers to an increase in drug deposition (deposited lung dose) arising out of, for example, the improved efficiency of drug delivery with a high efficiency nebulizer. In general, a high efficiency nebulizer will produce a drug cloud having a greater respirable fraction than a conventional nebulizer. While not wishing to be bound by theory, it is considered that a greater respirable fraction will permit greater lung deposition and concomitantly lower oropharyngeal deposition of the drug. In some embodiments, it is considered that reduced oropharyngeal deposition of drug will reduce local side effects, for example dry mouth.

“Deposited dose” or “deposited lung dose” is the amount of muscarinic antagonist deposited in the lung. The deposited dose or deposited lung dose may be expressed in absolute terms, for example the number of μg of API deposited in the lungs. The deposited lung dose may be expressed as a percentage of the nominal dose deposited in the lungs. The deposited lung dose may also be expressed in relative terms, for example comparing the mass of API deposited in the lungs with a high efficiency nebulizer to the mass of API deposited in the lungs with a conventional nebulizer.

“Cₘₐₓ^{HEN}” as used herein, refers to the maximum blood plasma concentration for a nominal dose of the active pharmaceutical ingredient (API) administered with a high efficiency nebulizer.

“Cₘₐₓ^{Conv}” as used herein, refers to the maximum blood plasma concentration for a nominal dose of the active pharmaceutical ingredient (API) administered with a conventional nebulizer.

“Enhanced pharmacokinetic profile” means an improvement in some pharmacokinetic parameter. Pharmacokinetic parameters that may be improved include, AUCₗₐₛₜ, AUC_{(0-∞)}Tₘₐₓ, and optionally a Cₘₐₓ. In some embodiments, the enhanced pharmacokinetic profile may be measured quantitatively by comparing a pharmacokinetic parameter obtained for a nominal dose of an active pharmaceutical ingredient (API) administered with one type of inhalation device (e.g. a high efficiency nebulizer) with the same pharmacokinetic parameter obtained with the same nominal dose of active pharmaceutical ingredient (API) administered with a different type of inhalation device.

“Blood plasma concentration” refers to the concentration of an active pharmaceutical ingredient (API) in the plasma component of blood of a subject or patient population.

“Respiratory condition,” as used herein, refers to a disease or condition that is physically manifested in the respiratory tract, including, but not limited to, chronic obstructive pulmonary disease (COPD), bronchitis, chronic bronchitis, emphysema, asthma, or reactive airway disorder (RAD).

“Patient” refers to the animal (especially mammal) or human being treated.

“Muscarinic antagonist” refers to antimuscarinic agents, which are compounds that have the ability to inhibit the action of the neurotransmitter acetylcholine by blocking its binding to muscarinic cholinergic receptors. These agents can be long-acting or short-acting. Long-acting muscarinic antagonists have a therapeutic effect lasting greater than about 6 hours. Some long-acting muscarinic antagonists include, but are not limited to, glycopyrrolate, tiotropium, aclidinium, trospium, darotropium, QAT 370, GSK 233705, GSK 573719, GSK 656398, TD4208, BEA 218 or a pharmaceutical acceptable derivative, salt, enantiomer, diastereomer, or racemic mixture thereof. Short-acting muscarinic antagonists have a therapeutic effect for less than about 6 hours. Some short-acting muscarinic antagonists include, but are not limited to, ipratropium, oxitropium, or a pharmaceutical acceptable derivative, salt, enantiomer, diastereomer, or racemic mixture thereof. In some embodiments, the “muscarinic antagonist” is glycopyrrolate, tiotropium, aclidinium, trospium, QAT370, GSK233705, GSK 656398, BEA2180, ipratropium, oxitropium, oxybutynin or a pharmaceutical acceptable derivative, salt, enantiomer, diastereomer, or a pharmaceutical acceptable derivative, salt, enantiomer, diastereomer, or racemic mixture thereof.

“Nebulizer,” as used herein, refers to a device that turns medications, compositions, formulations, suspensions, and mixtures, etc. into a fine mist for delivery to the lungs. Nebulizers may also be referred to as atomizers.

“Drug absorption” or simply “absorption” typically refers to the process of movement of drug from site of delivery of a drug across a barrier into a blood vessel or the site of action, e.g., a drug being absorbed in the pulmonary capillary beds of the alveoli.

[Tₘₐₓ^{HEN}] as used herein, refers to the amount of time necessary for a nominal dose of an active pharmaceutical ingredient (API) to attain maximum blood plasma concentration after administration with a high efficiency nebulizer.

[T1/2] Half-life: T1/2 in reference to the elimination rate of a drug, such as a muscarinic antagonist (e.g. glycopyrrolate) is the amount of time necessary for the drug's plasma concentration to drop to one-half of its initial plasma concentration.

[Tₘₐₓ^{Conv}] as used herein, refers to the amount of time necessary for a nominal dose of an active pharmaceutical ingredient (API) to attain maximum blood plasma concentration after administration with a conventional nebulizer.

The term “treat” and its grammatical variants (e.g. “to treat,” “treating,” and “treatment”) refer to administration of an active pharmaceutical ingredient to a patient with the purpose of ameliorating or reducing the incidence of one or more symptoms of a condition or disease state in the patient. Such symptoms may be chronic or acute; and such amelioration may be partial or complete. In the present context, treatment entails administering a muscarinic antagonist (optionally in combination with a beta 2-agonist) to a patient via a pulmonary inhalation route.

The term “prophylaxis” refers to administration of an active pharmaceutical ingredient to a patient with the purpose of reducing the occurrence or recurrence of one or more acute symptoms associated with a disease state in the patient. In the present context, prophylaxis entails administering a muscarinic antagonist (optionally in combination with a beta 2-agonist) to a patient via a pulmonary inhalation route. Thus, prophylaxis includes reduction in the occurrence or recurrence rate of acute exacerbations in chronic obstructive pulmonary disease (COPD). However, prophylaxis is not intended to include complete prevention of onset of a disease state in a patient who has not previously been identified as suffering from a pulmonary condition or disease; nor does prophylaxis include prevention of pulmonary cancer.

As used herein, a difference is “significant” if a person skilled in the art would recognize that the difference is probably real. In some embodiments, significance may be determined statistically—in which case two measured parameters may be referred to as statistically significant. In some embodiments, statistical significance may be quantified in terms of a stated confidence interval (CI), e.g. greater than 90%, greater than 95%, greater than 98%, etc. In some embodiments, statistical significance may be quantified in terms of a p value, e.g. less than 0.5, less than 0.1, less than 0.05, etc. The person skilled in the art will recognize these expressions of significance and will know how to apply them appropriately to the specific parameters that are being compared.

In some embodiments described herein an active pharmaceutical ingredient (API) is a muscarinic antagonist. In some embodiments, the API is substantially free of other bronchodilating agents, such as beta 2-agonists, like formoterol, salmeterol and salbutamol (albuterol). In this context, “substantially free of other bronchodilating agents” indicates that the solution contains no other bronchodilating agent or contains less than a quantity of another bronchodilating agent that would be sufficient to materially affect the properties of the muscarinic antagonist solution. In some embodiments, the API is a muscarinic antagonist (optionally in combination with a beta 2-agonist and/or in combination with an anti-inflammatory agent which could include a corticosteroid or a non-steroidal anti-inflammatory drug (NSAID)). In some embodiments, the API is free of other bronchodilating agents, such as beta 2-agonists, like formoterol, salmeterol and salbutamol (albuterol). In this context, “free of other bronchodilating agents” means that the solution contains no other bronchodilating agent than the recited muscarinic antagonist, or contains less than a detectable amount of the other bronchodilating agents.

Beta-2 adrenergic agonists are agents that mimic epinephrine in their interaction with β₂-adrenergic receptors. Thus, beta-2 adrenergic agonists are also referred to in the literature as beta-mimetics. A long-acting β₂ adrenergic agonist (LABA) is an active agent that has an effect similar to that of adrenaline, but with longer lasting effect (e.g. at least about 12 hr.) In the lung, LABAs stimulate adenlyate cyclase activity, closing calcium channels, and relaxing smooth muscle, thereby relieving bronchospasm. The following are generally classified as LABAs in the lung: bambuterol; bitolterol; carbuterol; clenbuterol; fenoterol; formoterol; hexoprenaline; ibuterol; indacaterol, pirbuterol; procaterol; reproterol; salmeterol; sulfonterol; tolubuterol; 4-hydroxy-7-[2-{[2-{[3-(2-phenylethoxy)propyl]sulfonyl}ethyl]-amino}ethyl]-2(3H-benzothiazolone; 1-(2-fluoro4-hydroxyphenyl)-2-[4-(1-benzimidazolyl)-2-methyl-2-butylamino]ethanol; 1-[3-(4-methoxybenzyl-amino)-4-hydroxyphenyl]-2-[4-(1-benzimidazolyl)-2-methyl-2-butylamino]ethanol; 1-[2H-5-hydroxy-3-oxo-4H-1,4-benzoxazin-8-yl]-2-[3-(4-N,N-dimethylaminophenyl)-2-methyl-2-propylamino]ethanol; 1-[2H-5-hydroxy-3-oxo-4H-1,4-benzoxazin-8-yl]-2-[3-(4-methoxyphenyl)-2-methyl-propylamino]ethanol; 1-[2H-5-hydroxy-3-oxo-4H-1,4-benzoxazin-8-yl]-2-[3-(4-n-butyloxyphenyl)-2-methyl-2-propylamino]ethanol; 1-[2H-5-hydroxy-3-oxo-4H-1,4-benzoxazin-8-yl]-2-{4-[3-(4-methoxyphenyl)-1, 2,4-triazol-3-yl]-2-methyl-2-butylamino}ethanol; 5-hydroxy-8-(1-hydroxy-2-isopropylaminobutyl)-2H-1,4-benzoxazin-3-(4H)-one; 1-(4-amino-3-chloro-5-trifluoromethylphenyl)-2-tert-butylamino)ethanol, or 1-(4-ethoxycarbonylamino-3-cyano-5-fluorophenyl)-2-(tert-butylamino)ethanol; or the racemates, enantiomers, diastereomers, or mixtures thereof, optionally in the form of their pharmacologically-compatible acid addition salts. In particular, formoterol may be present as the enantiomerically pure (at least about 90%) R,R-formoterol (or a suitable salt thereof), which is also referred to herein as arformoterol. As used herein “racemic formoterol” refers to the approximately 50:50 mixture of R,R-formoterol and its enantiomer S,S-formoterol. Salmeterol may be present as the enantiomerically pure (at least about 90%) R-salmeterol or as “racemic salmeterol,” which is an approximately 50:50 mixture of R-salmeterol and S-salmeterol or a suitable salt thereof.

Muscarinic Antagonists are agents that have the ability to inhibit the action of the neurotransmitter acetylcholine by blocking its binding to muscarinic cholinergic receptors. These agents can be long-acting or short-acting. Long-acting muscarinic antagonists (LAMAs) have a therapeutic effect lasting greater than about 6 hours. Some long-acting muscarinic antagonists include, but are not limited to, glycopyrrolate, R,R-glycopyrrolate, tiotropium, aclidinium, trospium, QAT 370, GSK, 233705, GSK 656398, BEA 218 or a pharmaceutical acceptable derivative, salt, enantiomer, diastereomer, or racemic mixture thereof. Short-acting muscarinic antagonists have a therapeutic effect for less than about 6 hours. Some short-acting muscarinic antagonists include, but are not limited to, ipratropium, oxitropium, or a pharmaceutical acceptable derivative, salt, enantiomer, diastereomer, or racemic mixture thereof. In some embodiments, the “muscarinic antagonist” is glycopyrrolate, tiotropium, aclidinium, trospium, QAT370, GSK233705, GSK 656398, BEA2180, ipratropium, oxitropium, oxybutynin or a pharmaceutical acceptable derivative, salt, enantiomer, diastereomer, or a pharmaceutical acceptable derivative, salt, enantiomer, diastereomer, or racemic mixture thereof. In some embodiments, the muscarinic antagonist is glycopyrrolate. In some embodiments, the muscarinic antagonist is racemic glycopyrrolate; in other embodiments the muscarinic antagonist is enriched in either the S,S- or R,R-enantiomer of glycopyrrolate. In some embodiments, the muscarinic antagonist is at least 55%, at least 60%, at least 70%, at least 80%, at least 90%, at least 95%, at least 98%, at least 99% or at least 99.5% enantiomerically pure R,R-glycopyrrolate.

Where a compound is mentioned herein without qualification of its physical form (e.g. enantiomer, salt and/or polymorphic form), the intended meaning is the compound in any of its known, possible forms.

“Monotherapy” refers to administration of an active pharmaceutical agent, e.g. a muscarinic antagonist as the sole active ingredient. This distinguishes monotherapy from combination therapy, in which two active pharmaceutical agents, e.g. a muscarinic antagonist and a LABA, are combined in a single therapeutic regime, e.g. by co-administration in a single dosage form, or by serial administration.

As used herein “combination” refers to a mixture or serially administered compositions. A mixture may be formed as a unit dose during the manufacturing process; a mixture may also be formed by combination of two separate unit doses prior to administration of the mixture to a patient. A combination may also refer to separate unit doses administered serially in a time frame that may be considered a single dosing event—e.g. less than about 30 minutes, less than about 20 minutes, or less than about 10 minutes.

A “standard dose” of a drug is either: (a) if the drug has been approved by a governmental body (such as the United States Food and Drug Administration), a government approved dose of the drug; or (b) if the drug has not been approved, a minimum therapeutically effective dose of the drug. A “minimum therapeutically effective dose” is the lowest dose administered with a conventional nebulizer that provides a therapeutic effect for a period of at least 12 hours, with acceptable side effects, in a patient population. For formoterol, the standard dose is 20 μg of formoterol administered as the fumarate salt by nebulization with a conventional nebulizer twice per day (B.I.D.) For arformoterol (R,R-formoterol), the standard dose is 15 μg of arformoterol administered as the tartrate salt with a conventional nebulizer twice per day (B.I.D.).

In some embodiments described herein an active pharmaceutical ingredient (API) is a LABA or a muscarinic antagonist in combination with a LABA, such as formoterol (racemate), arformoterol, salmeterol, clenbuterol, etc.

Some embodiments described herein provide a method of treating a patient having chronic obstructive pulmonary disease (COPD), comprising administering to the patient, with a high efficiency nebulizer, a dose of a long-acting beta 2-agonist (LABA) that produces a significantly improved therapeutic effect in the patient compared to administration of the same dose of the LABA with a conventional nebulizer. In some embodiments, administering the LABA with the high efficiency nebulizer results in significantly improved magnitude or duration of therapeutic effect, and/or significantly improved side effects, compared to administering the LABA with a conventional nebulizer, a metered dose inhaler, or a dry powder inhaler. In some embodiments, the dose of the LABA is an amount of the LABA that produces clinically meaningful bronchodilation for at least 24 hours when administered with a high efficiency nebulizer, wherein the same LABA produces significantly less than 24 hours (e.g. less than 20 hours, less than 18 hours, less than 16 hours or 12 hours or less) clinically meaningful bronchodilation when administered with a conventional nebulizer, a metered dose inhaler or a dry powder inhaler. In some embodiments, the clinically meaningful bronchodilation is an increase in trough FEV₁ of at least 10% or at least 100 mL above placebo. In some embodiments, the dose of the LABA is an amount of the LABA that produces clinically meaningful bronchodilation, with acceptable side effects, for at least 24 hours when administered with a high efficiency nebulizer, and wherein the same LABA produces significantly less than 24 hours (e.g. less than about 20 hours, less than about 18 hours, less than about 16 hours, or 12 hours or less) clinically meaningful bronchodilation, with acceptable side effects, when administered to the lungs with a conventional nebulizer, a metered dose inhaler or a dry powder inhaler. In some embodiments, wherein the LABA that is administered comprises formoterol, salmeterol, or a pharmaceutically acceptable enantiomer and/or salt thereof.

Some embodiments provide a method of treating a patient having chronic obstructive pulmonary disease (COPD), comprising administering to the patient a LABA with a high efficiency nebulizer that significantly improves the duration and/or magnitude of therapeutic effect of the LABA, while retaining acceptable side effects, compared to the same LABA administered with a conventional nebulizer, metered dose inhaler or dry powder inhaler. In some embodiments, the LABA administered with the high efficiency nebulizer results in clinically meaningful bronchodilation for at least 24 hours with acceptable side effects, and wherein the same LABA administered by a conventional nebulizer, metered dose inhaler or dry powder inhaler results in significantly less than 24 hours (e.g. less than about 20 hours, less than about 18 hours, less than about 16 hours, or 12 hours or less) of clinically meaningful bronchodilation with acceptable side effects. In some embodiments, the LABA is formoterol, salmeterol, or a pharmaceutically acceptable enantiomer and/or salt thereof.

In some embodiments, the formoterol dose is delivered in a fill volume of about 0.5 mL or less. In some embodiments, the formoterol dose is delivered in about 3 min. or less. In some embodiments, the formoterol is a 50:50 mixture of R,R-formoterol and S,S-formoterol. In some embodiments, the formoterol dose is less than about 10 μg.

In some embodiments, the formoterol dose is about 0.5 μg to about 8 μg, about 1 μg to about 8 μg, about 2 μg to about 8 μg, about 3 μg to about 8 μg, about 4 μg to about 8 μg, about 5 μg to about 8 μg, about 6 μg to about 8 μg, about 0.5 μg to about 6 μg, about 1 μg to about 6 μg, about 2 μg to about 6 μg, about 4 μg to about 6 μg, about 0.5 μg to about 5 μg, about 1 μg to about 5 μg, about 2 μg to about 5 μg, about 3 μg to about 5 μg, about 4 μg to about 5 μg, about 0.5 μg to about 4 μg, about 1 μg to about 4 μg, about 2 μg to about 4 μg, about 0.5 μg, about 1 μg, about 2 μg, about 3 μg, about 4 μg, about 5 μg, about 6 μg, about 7 μg, about 8 μg or about 9 μg.

In some embodiments, the formoterol is an enantiomerically enriched formoterol, which is greater than 90% enantiomerically pure R,R-formoterol. In some embodiments, the enantiomerically enriched formoterol is greater than 92%, greater than 93%, greater than 94%, greater than 95%, greater than 96%, greater than 97%, greater than 98%, about 95%, about 96%, about 97%, about 98%, about 99%, about 99.5%, about 99.6%, about 99.7%, about 99.8% or about 99.9% of R,R-formoterol.

In some embodiments, the formoterol dose is less than about 7.5 μg of enantiomerically pure R,R-formoterol. In some embodiments, the formoterol dose is about 0.25 μg to about 7 μg, about 0.5 μg to about 7 μg, about 1 μg to about 7 μg, about 2 μg to about 7 μg, about 3 μg to about 7 μg, about 4 μg to about 7 μg, 0.25 μg to about 6 μg, about 0.5 μg to about 6 μg, about 1 μg to about 6 μg, about 2 μg to about 6 μg, about 3 μg to about 6 μg, about 4 μg to about 6 μg, about 0.25 μg to about 5 μg, about 0.5 μg to about 5 μg, about 1 μg to about 5 μg, about 2 μg to about 5 μg, about 3 μg to about 5 μg, about 4 μg to about 5 μg, about 0.25 μg to about 4 μg, about 0.5 μg to about 4 μg, about 1 μg to about 4 μg, about 2 μg to about 4 μg, about 0.25 μg to about 2 μg, about 0.5 μg to about 2 μg, about 1 μg to about 2 μg, about 0.25 μg to about 1 μg, about 0.25 μg, about 0.5 μg, about 1 μg, about 2 μg, about 3 μg, about 4 μg, about 5 μg or about 6 μg of R,R-formoterol.

Some embodiments provide a method of treating a patient having chronic obstructive pulmonary disease (COPD), comprising administering to the patient with a high efficiency nebulizer an amount of formoterol sufficient to produce a therapeutic effect with acceptable side effects for at least 24 hours.

Some embodiments provide a method of treating a patient having chronic obstructive pulmonary disease (COPD), comprising administering to the patient with a high efficiency nebulizer an amount of formoterol or a combination of glycopyrrolate and formoterol sufficient to produce a therapeutic effect with acceptable side effects for at least 24 hours. In some embodiments the duration of therapeutic effect is at least 28 hours, at least 30 hours, at least 32 hours or at least 36 hours. In some embodiments, the side effects are reduced compared to: (a) an approved dose of formoterol; (b) a minimally effective dose of glycopyrrolate; or (c) both (a) and (b). In some embodiments, the reduced side effects include one or more of the following: (a) side effects associated with formoterol; (b) side effects associated with glycopyrrolate. In some embodiments, the reduced side effects include at least one of the following: airway hyperreactivity (hypersensitivity), angina, anorexia, anxiety, backaches, blurred vision, bradycardia, central stimulation, chest discomfort (e.g. chest pain), coughing, diarrhea, dizziness, drowsiness, drying or irritation of the oropharynx (such as dry mouth (xerostomia)), dyspnea, excitement, fatigue, flushing, hand tremors, headache, hoarseness, hypotension and palpitations, impotence, increased heart rate, insomnia, mental confusion, muscle cramps, muscle tremors, nausea, nervousness, palpitations, sweating, tachycardia, unusual taste, urinary hesitancy and retention, vertigo, vomiting, weakness, and wheezing. In some embodiments, the nominal dose of glycopyrrolate is less than about 100 μg to about 1600 μg, e.g. about 25 μg to about 500 μg or about 50 μg to about 300 μg. In some embodiments, the nominal dose. In some embodiments, the nominal dose of formoterol is about 1 to about 20 μg. In some embodiments, the formoterol is a 50:50 mixture of R,R- and S,S-formoterol or at least 90% enantiomerically pure R,R-formoterol. In some embodiments, the formoterol is some mixture of R,R- and S,S-formoterol of a ratio between 100:0 and 0:100. In some embodiments, the mixture is at least about 60%, at least about 70%, at least about 80%, at least about 90%, at least about 95%, at least about 98%, at least about 99% or at least about 99.5% enantiomerically pure R,R-formoterol. In some embodiments, the combination is delivered with a high efficiency nebulizer. In some embodiments, the combination has a fill volume of ˜0.5 mL or less. In some embodiments, the combination is delivered in about 3 minutes or less. In some embodiments, the combination is delivered with a conventional nebulizer. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments provide a method of treating a patient having a respiratory condition, comprising administering to the patient with a high efficiency nebulizer a reduced dose of a long acting beta agonist (LABA), wherein said reduced dose of LABA is less than half of a minimum effective therapeutic dose of said LABA administered with a conventional nebulizer, and which provides (a) similar magnitude of therapeutic effect; (b) similar duration of therapeutic effect; or both (a) and (b), compared with administration of the minimum effective therapeutic dose of said LABA with a conventional nebulizer.

Some embodiments described herein provide a method of treating a patient having chronic obstructive pulmonary disease (COPD), comprising administering to the patient with a nebulizer a combination of a nominal dose of glycopyrrolate and a nominal dose of formoterol, wherein said administration produces: (a) an increased magnitude of therapeutic effect; and (b) reduced side effects, as compared to administration, with the same nebulizer, of: (1) said nominal dose of glycopyrrolate alone; or (2) said nominal dose of formoterol alone.

Some embodiments described herein provide a method of treating a patient having chronic obstructive pulmonary disease (COPD), comprising administering to the patient with a high efficiency nebulizer a reduced dose of a long-acting beta 2-agonist (LABA), wherein said reduced dose of LABA is less than half of an approved therapeutic dose of LABA administered with a conventional nebulizer, a metered dose inhaler, or a dry powder inhaler and wherein the reduced dose of LABA provides (a) similar magnitude of therapeutic effect; (b) similar duration of therapeutic effect; or both (a) and (b), compared with administration of the approved therapeutic dose of LABA with a conventional nebulizer, a metered dose inhaler, or a dry powder inhaler. In some embodiments, the LABA is formoterol, salmeterol, or a pharmaceutically acceptable enantiomer and/or salt thereof. In some embodiments, administration of the LABA with the high efficiency nebulizer results in reduced side effects compared to the approved therapeutic dose of the LABA administered with a conventional nebulizer, a metered dose inhaler, or a dry powder inhaler. In some embodiments, the LABA is formoterol, or a pharmaceutically acceptable salt thereof, and is administered at a dose of less than about 10 μg. In some embodiments, the LABA is R,R-formoterol, or a pharmaceutically acceptable salt thereof, and is administered at a dose of less than about 7.5 μg of enantiomerically pure R,R-formoterol. In some embodiments, the LABA is salmeterol, or a pharmaceutically acceptable salt thereof, and is administered at a dose of less than about 25 μg.

Some embodiments described herein provide a method of treating a patient having a respiratory condition, comprising administering to the patient with a high efficiency nebulizer a nominal, respirable, or deposited dose of LABA, wherein said administration provides: (i) an increased magnitude of therapeutic effect; (ii) an increased duration of therapeutic effect; and/or (iii) reduced side effects, as compared to administration of the same nominal, respirable, or deposited dose of LABA with a conventional nebulizer. In some embodiments, the LABA dose is delivered in a fill volume of about 0.5 mL or less. In some embodiments, the LABA dose is delivered in about 3 min. or less. In some embodiments, the LABA is a 50:50 mixture of R,R-formoterol and S,S-formoterol. In some embodiments, the formoterol is an enantiomerically enriched formoterol, which is greater than 90% enantiomerically pure R,R-formoterol (arformoterol). In some embodiments, the LABA is selected from the group consisting of formoterol (50:50 mixture of R,R- and S,S-formoterol), salmeterol (50:50 mixture of R- and S-salmeterol), R-salmeterol, R,R-formoterol, bambuterol, clenbuterol or indacaterol, or a pharmaceutically acceptable salt thereof. In some embodiments, the respiratory condition.

Some embodiments described herein provide a method of treating a patient having chronic obstructive pulmonary disease (COPD), comprising administering to the patient with a high efficiency nebulizer a dose of a long-acting beta 2-agonist (LABA), wherein said administration provides: (i) an increased magnitude of therapeutic effect; (ii) an increased duration of therapeutic effect; and/or (iii) reduced side effects, as compared to administration of a dose of the LABA, with a conventional nebulizer, that achieves the same respirable or deposited dose as is achieved with the high efficiency nebulizer. In some embodiments, the LABA is formoterol, salmeterol, or a pharmaceutically acceptable enantiomer and/or salt thereof. In some embodiments, there is provided a method of treating a patient having chronic obstructive pulmonary disease (COPD), comprising administering to the patient with a high efficiency nebulizer a dose of long-acting beta 2-agonist (LABA), wherein said administration provides substantially the same magnitude and duration of therapeutic effect, and reduced side effects, as compared to administration of a dose of the LABA, with a conventional nebulizer, metered dose inhaler or dry powder inhaler that is necessary to achieve the same respirable or deposited dose as is achieved with the high efficiency nebulizer. In some embodiments, the LABA is formoterol, salmeterol, indacaterol, or a pharmaceutically acceptable enantiomer and/or salt thereof.

Some embodiments described herein provide a method of treating a patient having chronic obstructive pulmonary disease (COPD), comprising administering to the patient, with a high efficiency nebulizer, a dose of a combination of an amount of a long-acting beta 2-agonist (LABA) and an amount of a long-acting muscarinic antagonist (LAMA), wherein the dose of the combination is effective to produce a significantly improved therapeutic effect in the patient compared to administration of the LABA with a nebulizer as a monotherapy, and compared to administration of the LAMA with a nebulizer as a monotherapy. In some embodiments, the method comprises administering the dose of the combination with the high efficiency nebulizer results in significantly improved magnitude or duration of therapeutic effect, and/or significantly improved side effects, compared to administering the LABA with a nebulizer as a monotherapy and compared to administering the LAMA with a nebulizer as a monotherapy. In some embodiments, the dose of the combination refers to the nominal, respirable or deposited dose of the combination. In some embodiments, the dose of the combination is an amount of the LABA that produces clinically meaningful bronchodilation with acceptable side effects for significantly less than 24 hours when administered with a nebulizer and/or an amount of the LAMA that produces clinically meaningful bronchodilation with acceptable side effects for significantly less than 24 hours when administered with a nebulizer, wherein the dose of the combination produces clinically meaningful bronchodilation with acceptable side effects of 24 hours or more when administered with a high efficiency nebulizer. In some embodiments, the clinically meaningful bronchodilation is an increase in trough FEV₁ of at least 10% or 100 mL above placebo. In some embodiments, the LABA is formoterol, salmeterol, indacaterol, or a pharmaceutically acceptable enantiomer and/or salt thereof. In some embodiments, the LAMA is glycopyrrolate or a pharmaceutically acceptable enantiomer and/or salt thereof. In some embodiments, the LABA is formoterol or a pharmaceutically acceptable enantiomer and/or salt thereof and the LAMA is glycopyrrolate or a pharmaceutically acceptable enantiomer and/or salt thereof. In some embodiments, said administration produces: (a) an increased duration of therapeutic effect; and (b) reduced, similar or acceptable side effects, as compared to administration, with the same nebulizer, of: (1) said nominal dose of glycopyrrolate alone; and (2) said nominal dose of formoterol alone. In some embodiments, said administration results in a duration of therapeutic effect greater than about 20 hr, greater than about 22 hr or at least about 24 hr. In some embodiments the duration of therapeutic effect is at least 12, 18, 20, 24, 28, 30, 32 or 36 hr. In some embodiments, the increased magnitude of effect is greater than 5% higher than provided by: (1) said nominal dose of glycopyrrolate alone; and (2) said nominal dose of formoterol alone. In some embodiments, the combination is administered with a high efficiency nebulizer. In some embodiments, the combination is administered in a fill volume of about 0.5 mL or less. In some embodiments, the combination is administered in about 3 minutes or less. In some embodiments, the combination is administered with a conventional nebulizer. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having chronic obstructive pulmonary disease (COPD), comprising administering to the patient, with a high efficiency nebulizer, a dose of a combination of an amount of a long-acting beta 2-agonist (LABA) and an amount of a long-acting muscarinic antagonist (LAMA), wherein the dose of the combination is effective to produce a significantly improved therapeutic effect in the patient compared to administration of the LABA with a nebulizer, metered dose inhaler, or dry powder inhaler as a monotherapy, and compared to administration of the LAMA with a nebulizer, metered dose inhaler, or dry powder inhaler as a monotherapy. In some embodiments, the method comprises administering the dose of the combination with the high efficiency nebulizer results in significantly improved magnitude or duration of therapeutic effect, and/or significantly improved side effects, compared to administering the LABA with a nebulizer, metered dose inhaler, or dry powder inhaler as a monotherapy and compared to administering the LAMA with a nebulizer as a monotherapy. In some embodiments, the dose of the combination refers to the nominal, respirable or deposited dose of the combination. In some embodiments, the dose of the combination is an amount of the LABA that produces clinically meaningful bronchodilation with acceptable side effects for significantly less than 24 hours when administered with a nebulizer metered dose inhaler, or dry powder inhaler and/or an amount of the LAMA that produces clinically meaningful bronchodilation with acceptable side effects for significantly less than 24 hours when administered with a nebulizer, wherein the dose of the combination produces clinically meaningful bronchodilation with acceptable side effects of 24 hours or more when administered with a high efficiency nebulizer. In some embodiments, the clinically meaningful bronchodilation is an increase in trough FEV₁ of at least 10% or 100 mL above placebo. In some embodiments, the LABA is formoterol, salmeterol, indacaterol, or a pharmaceutically acceptable enantiomer and/or salt thereof. In some embodiments, the LAMA is glycopyrrolate or a pharmaceutically acceptable enantiomer and/or salt thereof. In some embodiments, the LABA is salmeterol, indacaterol, or a pharmaceutically acceptable enantiomer and/or salt thereof and the LAMA is glycopyrrolate or a pharmaceutically acceptable enantiomer and/or salt thereof.

In some embodiments, said administration produces: (a) similar or increased magnitude and/or duration of therapeutic effect; and (b) reduced side effects, compared to administration, with the same nebulizer, of: (1) said standard dose of glycopyrrolate alone; and (2) said standard dose of formoterol alone. In some embodiments, said administration produces: (a) similar or increased magnitude and/or duration of therapeutic effect; and (b) reduced side effects, compared to administration, with the same nebulizer, of: (1) said standard dose of glycopyrrolate alone; and (3) a combination of said standard dose of glycopyrrolate and said standard dose of formoterol. In some embodiments, said administration produces: (a) similar or increased magnitude and/or duration of therapeutic effect; and (b) reduced side effects, compared to administration, with the same nebulizer, of: (1) said standard dose of glycopyrrolate alone; and (2) said standard dose of formoterol alone; and (3) a combination of said standard dose of glycopyrrolate and said standard dose of formoterol.

Some embodiments described herein provide a method of treating a patient having COPD, comprising administering to the patient with a high efficiency nebulizer a combination of (A) a reduced dose of glycopyrrolate; and/or (B) a reduced dose of formoterol, wherein (I) said reduced dose of glycopyrrolate is significantly less than a standard dose of glycopyrrolate; and (II) said reduced dose of formoterol is significantly less than a standard dose of formoterol, and wherein said administration produces: (a) increased magnitude and/or duration of therapeutic effect; and (b) reduced side effects, compared to administration, with a conventional nebulizer, of: (1) said standard dose of glycopyrrolate alone; or (2) said standard dose of formoterol alone.

Some embodiments described herein provide a method of treating a patient having COPD, comprising administering to the patient with a high efficiency nebulizer a combination of a dose of glycopyrrolate and a dose of formoterol, wherein said administration produces: (a) similar or increased magnitude and/or duration of therapeutic effect; and (b) reduced side effects, compared to administration, in a conventional nebulizer, of: (1) the equivalent respirable dose of glycopyrrolate; 2) the equivalent respirable dose of formoterol; or 3) the combination of the equivalent respirable doses of glycopyrrolate and formoterol. In some embodiments, said administration produces: (a) similar or increased magnitude and/or duration of therapeutic effect; and (b) reduced side effects, compared to administration, with the same nebulizer, of: (1) said standard dose of glycopyrrolate alone; and (2) said standard dose of formoterol alone.

Some embodiments described herein provide a method of treating a patient having chronic obstructive pulmonary disease (COPD), comprising administering to the patient an amount of a combination of a LAMA and a LABA sufficient to produce a therapeutic effect with acceptable side effects for at least 24 hours. In some embodiments, the side effects are reduced compared to: (a) a minimum therapeutically effective dose of said LABA; (b) a minimum therapeutically effective dose of said LAMA; or (c) both (a) and (b). In some embodiments, the reduced side effects include one or more of the following: (a) side effects associated with a LABA; (b) side effects associated with a LAMA; or (c) both (a) and (b). In some embodiments, the reduced side effects include at least one or more of the following: airway hyperreactivity (hypersensitivity), angina, anorexia, anxiety, backaches, blurred vision, bradycardia, central stimulation, chest discomfort (e.g. chest pain), coughing, diarrhea, dizziness, drowsiness, drying or irritation of the oropharynx (such as dry mouth (xerostomia)), dyspnea, excitement, fatigue, flushing, hand tremors, headache, hoarseness, hypotension and palpitations, impotence, increased heart rate, insomnia, mental confusion, muscle cramps, muscle tremors, nausea, nervousness, palpitations, sweating, tachycardia, unusual taste, urinary hesitancy and retention, vertigo, vomiting, weakness, and wheezing. In some embodiments, the combination is delivered with a high efficiency nebulizer. In some embodiments, the combination has a fill volume of ˜0.5 mL or less. In some embodiments, the combination is delivered in about 3 minutes or less. In some embodiments, the combination is delivered with a conventional nebulizer. In some embodiments, (a) said LAMA is glycopyrrolate, tiotropium, aclidinium, trospium, QAT370, GSK233705, GSK 656398, or BEA2180, or a pharmaceutically acceptable derivative, salt, enantiomer, diastereomer, or racemic mixture thereof; and (b) said LABA is formoterol (such as racemic formoterol, i.e. a 50:50 mixture of R,R- and S,S-formoterol), salmeterol (50:50 mixture of R- and S-salmeterol), R-salmeterol, R,R-formoterol, bambuterol, clenbuterol or indacaterol, or a pharmaceutically acceptable derivative, salt, enantiomer, diastereomer, or racemic mixture thereof. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments provided herein provide a method of treating a patient having chronic obstructive pulmonary disease (COPD), comprising administering to the patient with a nebulizer a combination of a nominal dose of a LAMA and a nominal dose of a LABA, wherein said administration produces: (a) an increased magnitude of therapeutic effect; and (b) reduced side effects, as compared to administration, with the same nebulizer, of: (1) said nominal dose of said LAMA alone; or (2) said nominal dose of said LABA alone. In some embodiments, said administration produces: (a) an increased magnitude of therapeutic effect; and (b) reduced side effects, as compared to administration, with the same nebulizer, of: (1) said nominal dose of said LAMA alone; and (2) said nominal dose of said LABA alone. In some embodiments, the magnitude of therapeutic effect is compared at about 12 hr post delivery. In some embodiments, the duration of therapeutic effect is at least about 12 hr. In some embodiments, the increased magnitude of effect is greater than 5% higher than provided by: (1) said nominal dose of said LAMA alone; and (2) said nominal dose of said LABA alone. In some embodiments, the combination is administered with a high efficiency nebulizer. In some embodiments, the combination is administered in a fill volume of about 0.5 mL or less. In some embodiments, the combination is administered in about 3 minutes or less. In some embodiments, the combination is administered with a conventional nebulizer. In some embodiments, (a) said LAMA is glycopyrrolate, tiotropium, aclidinium, trospium, QAT370, GSK233705, GSK 656398, or BEA2180, or a pharmaceutically acceptable derivative, salt, enantiomer, diastereomer, or racemic mixture thereof; and (b) said LABA is formoterol (such as racemic formoterol, i.e. a 50:50 mixture of R,R- and S,S-formoterol), salmeterol (50:50 mixture of R- and S-salmeterol), R-salmeterol, R,R-formoterol, bambuterol, clenbuterol or indacaterol, or a pharmaceutically acceptable derivative, salt, enantiomer, diastereomer, or racemic mixture thereof. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having COPD, comprising administering to the patient with a nebulizer a combination of a nominal dose of a LAMA and a nominal dose of a LABA, wherein said administration produces: (a) an increased duration of therapeutic effect; and (b) reduced, similar or acceptable side effects, as compared to administration, with the same nebulizer, of: (1) said nominal dose of said LAMA alone; or (2) said nominal dose of said LABA alone. In some embodiments, said administration produces: (a) an increased duration of therapeutic effect; and (b) reduced, similar or acceptable side effects, as compared to administration, with the same nebulizer, of: (1) said nominal dose of said LAMA alone; and (2) said nominal dose of said LABA alone. In some embodiments, said administration results in a duration of therapeutic effect greater than about 20 hr, greater than about 22 hr or at least about 24 hr. In some embodiments, the increased magnitude of effect is greater than 5% higher than provided by: (1) said nominal dose of said LAMA alone; and (2) said nominal dose of said LABA alone. In some embodiments, the combination is administered with a high efficiency nebulizer. In some embodiments, the combination is administered in a fill volume of about 0.5 mL or less. In some embodiments, the combination is administered in about 3 minutes or less. In some embodiments, the combination is administered with a conventional nebulizer. In some embodiments, (a) said LAMA is glycopyrrolate, tiotropium, aclidinium, trospium, QAT370, GSK233705, GSK 656398, or BEA2180, or a pharmaceutically acceptable derivative, salt, enantiomer, diastereomer, or racemic mixture thereof; and (b) said LABA is formoterol (such as racemic formoterol, i.e. a 50:50 mixture of R,R- and S,S-formoterol), salmeterol (50:50 mixture of R- and S-salmeterol), R-salmeterol, R,R-formoterol, bambuterol, clenbuterol or indacaterol, or a pharmaceutically acceptable derivative, salt, enantiomer, diastereomer, or racemic mixture thereof. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having COPD, comprising administering to the patient with a nebulizer a combination of (A) a nominal dose of a LAMA and (B) a nominal dose of a LABA, wherein at least one of the nominal doses of said LAMA or said LABA is significantly less than a standard dose; and wherein said administration produces: (a) similar or increased magnitude and/or duration of therapeutic effect; and (b) reduced side effects, compared to administration, with the same nebulizer, of: (1) said standard dose of said LAMA alone; or (2) said standard dose of said LABA alone; or (3) a combination of said standard dose of said LAMA and said standard dose of said LABA.

Some embodiments described herein provide a method of treating a patient having COPD, comprising administering to the patient with a high efficiency nebulizer a combination of: (A) a reduced dose of said LAMA; and/or (B) a reduced dose of said LABA, wherein (I) said reduced dose of said LAMA is significantly less than a standard dose of said LAMA; and (II) said reduced dose of said LABA is significantly less than a standard dose of said LABA, and wherein said administration produces: (a) increased magnitude and/or duration of therapeutic effect; and (b) reduced side effects, compared to administration, with a conventional nebulizer, of: (1) said standard dose of said LAMA alone; or (2) said standard dose of said LABA alone. In some embodiments, said administration produces: (a) similar or increased magnitude and/or duration of therapeutic effect; and (b) reduced side effects, compared to administration, with the same nebulizer, of: (1) said standard dose of said LAMA alone; and (2) said standard dose of said LABA alone. In some embodiments, said administration produces: (a) similar or increased magnitude and/or duration of therapeutic effect; and (b) reduced side effects, compared to administration, with the same nebulizer, of: (1) said standard dose of said LAMA alone; and (3) a combination of said standard dose of said LAMA and said standard dose of said LABA. In some embodiments, said administration produces: (a) similar or increased magnitude and/or duration of therapeutic effect; and (b) reduced side effects, compared to administration, with the same nebulizer, of: (1) said standard dose of said LAMA alone; and (2) said standard dose of said LABA alone; and (3) a combination of said standard dose of said LAMA and said standard dose of said LABA.

Some embodiments described herein provide a method of treating a patient having COPD, comprising administering to the patient with a high efficiency nebulizer a combination of a dose of said LAMA and a dose of said LABA, wherein said administration produces: (a) similar or increased magnitude and/or duration of therapeutic effect; and (b) reduced side effects, compared to administration, in a conventional nebulizer, of: (1) the equivalent respirable dose of said LAMA; 2) the equivalent respirable dose of said LABA; or 3) the combination of the equivalent respirable doses of said LAMA and LABA. In some embodiments, said administration produces: (a) similar or increased magnitude and/or duration of therapeutic effect; and (b) reduced side effects, compared to administration, with the same nebulizer, of: (1) said standard dose of said LAMA alone; and (2) said standard dose of said LABA alone. In some embodiments, said administration produces: (a) similar or increased magnitude and/or duration of therapeutic effect; and (b) reduced side effects, compared to administration, with the same nebulizer, of: (1) said standard dose of said LAMA alone; and (3) a combination of said standard dose of said LAMA and said standard dose of said LABA. In some embodiments, said administration produces: (a) similar or increased magnitude and/or duration of therapeutic effect; and (b) reduced side effects, compared to administration, with the same nebulizer, of: (1) said standard dose of said LAMA alone; and (2) said standard dose of said LABA alone; and (3) a combination of said standard dose of said LAMA and said standard dose of said LABA. In some embodiments, the nominal dose of said LABA is significantly less than a standard dose of said LABA and the standard dose of said LABA is a government approved dose of said LABA administered with the same nebulizer. In some embodiments, the nominal dose of said LAMA is significantly less than a standard dose of said LAMA and the standard dose of said LAMA is a minimum effective therapeutic dose of said LAMA administered with the same nebulizer. In some embodiments, the nominal dose of said LABA is significantly less than a standard dose of said LABA and the standard dose of said LABA is a government approved dose of said LABA administered with the same nebulizer; and wherein the nominal dose of said LAMA is significantly less than a standard dose of said LAMA and the standard dose of said LAMA is a minimum effective therapeutic dose of said LAMA administered with the same nebulizer. In some embodiments, the duration of therapeutic effect is at least about 20 hr, at least about 22 hr or at least about 24 hr.

Some embodiments described herein provide a method of treating a patient having chronic obstructive pulmonary disease (COPD), comprising administering to the patient an amount of a combination of glycopyrrolate and formoterol sufficient to produce a therapeutic effect with reduced side effects for at least 24 hours, wherein the side effects are reduced compared to: (a) an approved dose of formoterol; (b) a minimally effective dose of glycopyrrolate; or (c) both (a) and (b). In some embodiments, the reduced side effects include one or more of the following: (a) side effects associated with formoterol; (b) side effects associated with glycopyrrolate. In some embodiments, the reduced side effects include at least one or more of the following: airway hyperreactivity (hypersensitivity), angina, anorexia, anxiety, backaches, blurred vision, bradycardia, central stimulation, chest discomfort (e.g. chest pain), coughing, diarrhea, dizziness, drowsiness, drying or irritation of the oropharynx (such as dry mouth (xerostomia)), dyspnea, excitement, fatigue, flushing, hand tremors, headache, hoarseness, hypotension and palpitations, impotence, increased heart rate, insomnia, mental confusion, muscle cramps, muscle tremors, nausea, nervousness, palpitations, sweating, tachycardia, unusual taste, urinary hesitancy and retention, vertigo, vomiting, weakness, and wheezing. In some embodiments, the nominal dose of glycopyrrolate is less than about 100 μg to about 1600 μg. In some embodiments, the nominal dose of formoterol is about 1 to about 20 μg.

In some embodiments, the formoterol dose is less than about 7.5 μg of enantiomerically pure R,R-formoterol. In some embodiments, the formoterol dose is about 0.25 μg to about 7 μg, about 0.5 μg to about 7 μg, about 1 μg to about 7 μg, about 2 μg to about 7 μg, about 3 μg to about 7 μg, about 4 μg to about 7 μg, 0.25 μg to about 6 μg, about 0.5 μg to about 6 μg, about 1 μg to about 6 μg, about 2 μg to about 6 μg, about 3 μg to about 6 μg, about 4 μg to about 6 μg, about 0.25 μg to about 5 μg, about 0.5 μg to about 5 μg, about 1 μg to about 5 μg, about 2 μg to about 5 μg, about 3 μg to about 5 μg, about 4 μg to about 5 μg, about 0.25 μg to about 4 μg, about 0.5 μg to about 4 μg, about 1 μg to about 4 μg, about 2 μg to about 4 μg, about 0.25 μg to about 2 μg, about 0.5 μg to about 2 μg, about 1 μg to about 2 μg, about 0.25 μg to about 1 μg, about 0.25 μg, about 0.5 μg, about 1 μg, about 2 μg, about 3 μg, about 4 μg, about 5 μg or about 6 μg of R,R-formoterol.

In some embodiments, the formoterol is a 50:50 mixture of R,R- and S,S-formoterol or at least 90% enantiomerically pure R,R-formoterol. In some embodiments, the combination is delivered with a high efficiency nebulizer. In some embodiments, the combination has a fill volume of ˜0.5 mL or less. In some embodiments, the combination is delivered in about 3 minutes or less. In some embodiments, the combination is delivered with a conventional nebulizer. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having chronic obstructive pulmonary disease (COPD), comprising administering to the patient with a nebulizer a combination of a nominal dose of glycopyrrolate and a nominal dose of formoterol, wherein said administration produces: (a) an increased magnitude of therapeutic effect; and (b) reduced side effects, as compared to administration, with the same nebulizer, of: (1) said nominal dose of glycopyrrolate alone; or (2) said nominal dose of formoterol alone, and wherein said administration produces. In some embodiments, the magnitude of therapeutic effect is compared at about 12 hr post delivery. In some embodiments, the duration of therapeutic effect is at least about 12 hr, at least about 18 hr, at least about 20 hr or at least about 24 hr. In some embodiments, the increased magnitude of effect is greater than 5% higher than provided by: (1) said nominal dose of glycopyrrolate alone; and (2) said nominal dose of formoterol alone. In some embodiments, the combination is administered with a high efficiency nebulizer. In some embodiments, the combination has a fill volume of about 0.5 mL or less. In some embodiments, the combination is administered in about 3 minutes or less. In some embodiments, the combination is administered with a conventional nebulizer. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having COPD, comprising administering to the patient with a nebulizer a combination of a nominal dose of glycopyrrolate and a nominal dose of formoterol, wherein said administration produces: (a) an increased duration of therapeutic effect; and (b) reduced, similar or acceptable side effects, as compared to administration, with the same nebulizer, of: (1) said nominal dose of glycopyrrolate alone; or (2) said nominal dose of formoterol alone. In some embodiments, said administration produces a duration of therapeutic effect greater than about 20 hr, greater than about 22 hr or at least about 24 hr. In some embodiments, the increased magnitude of effect is greater than 5% higher than provided by: (1) said nominal dose of glycopyrrolate alone; and (2) said nominal dose of formoterol alone. In some embodiments, the combination is administered with a high efficiency nebulizer. In some embodiments, the combination has a fill volume of about 0.5 mL or less. In some embodiments, the combination is administered in about 3 minutes or less. In some embodiments, the combination is administered with a conventional nebulizer. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having COPD, comprising administering to the patient with a nebulizer a combination of (A) a nominal dose of glycopyrrolate and (B) a nominal dose of formoterol, wherein at least one of the nominal doses of glycopyrrolate or formoterol is significantly less than a standard dose; and wherein said administration produces: (a) similar or increased magnitude and/or duration of therapeutic effect; and (b) reduced side effects, compared to administration, with the same nebulizer, of: (1) said standard dose of glycopyrrolate alone; (2) said standard dose of formoterol alone; and (3) a combination of said standard dose of glycopyrrolate and said standard dose of formoterol. In some embodiments, the reduced side effects include at least one or more of the following: airway hyperreactivity (hypersensitivity), angina, anorexia, anxiety, backaches, blurred vision, bradycardia, central stimulation, chest discomfort (e.g. chest pain), coughing, diarrhea, dizziness, drowsiness, drying or irritation of the oropharynx (such as dry mouth (xerostomia)), dyspnea, excitement, fatigue, flushing, hand tremors, headache, hoarseness, hypotension and palpitations, impotence, increased heart rate, insomnia, mental confusion, muscle cramps, muscle tremors, nausea, nervousness, palpitations, sweating, tachycardia, unusual taste, urinary hesitancy and retention, vertigo, vomiting, weakness, and wheezing.

In some embodiments, the dose of formoterol, glycopyrrolate or both is less than about 75% of the standard dose. In some embodiments, the dose of formoterol, glycopyrrolate or both is less than about 65%, 60%, 55%, 50%, 45%, 40%, 35%, 30%, 25%, 20% or 15% of the standard dose. In some embodiments, the combination is administered with a high efficiency nebulizer. In some embodiments, the combination has a fill volume of about 0.5 mL or less. In some embodiments, the combination is administered in significantly less than about 3 min. In some embodiments, the combination is administered with a conventional nebulizer. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Some embodiments described herein provide a method of treating a patient having COPD, comprising administering to the patient with a high efficiency nebulizer a combination of (A) a reduced dose of glycopyrrolate; and/or (B) a reduced dose of formoterol, wherein (I) said reduced dose of glycopyrrolate is significantly less than a standard dose of glycopyrrolate; and (II) said reduced dose of formoterol is significantly less than a standard dose of formoterol, and wherein said administration produces: (a) increased magnitude and/or duration of therapeutic effect; and (b) reduced side effects, compared to administration, with a conventional nebulizer, of: (1) said standard dose of glycopyrrolate alone; and (2) said standard dose of formoterol alone. In some embodiments, the nominal dose of formoterol is significantly less than a standard dose of formoterol and the standard dose of formoterol is a government approved dose of formoterol administered with the same nebulizer. In some embodiments, the nominal dose of glycopyrrolate is significantly less than a standard dose of glycopyrrolate and the standard dose of glycopyrrolate is a minimum effective therapeutic dose of glycopyrrolate administered with the same nebulizer.

Some embodiments described herein provide a method of treating a patient having COPD, comprising administering to the patient with a high efficiency nebulizer a combination of a dose of glycopyrrolate and a dose of formoterol, wherein said administration produces: (a) similar or increased magnitude and/or duration of therapeutic effect; and (b) reduced side effects, compared to administration, in a conventional nebulizer, of: (1) the equivalent respirable dose of glycopyrrolate; 2) the equivalent respirable dose of formoterol; and 3) the combination of the equivalent respirable doses of glycopyrrolate and formoterol. In some embodiments, the nominal dose of glycopyrrolate is significantly less than a standard dose of formoterol and the standard dose of formoterol is a government approved dose of formoterol administered with the same nebulizer. In some embodiments, the nominal dose of glycopyrrolate is significantly less than a standard dose of glycopyrrolate and the standard dose of glycopyrrolate is a minimum effective therapeutic dose of glycopyrrolate administered with the same nebulizer. In some embodiments, the nominal dose of glycopyrrolate is significantly less than a standard dose of formoterol and the standard dose of formoterol is a government approved dose of formoterol administered with the same nebulizer; and wherein the nominal dose of glycopyrrolate is significantly less than a standard dose of glycopyrrolate and the standard dose of glycopyrrolate is a minimum effective therapeutic dose of glycopyrrolate administered with the same nebulizer. In some embodiments, the duration of therapeutic effect is at least about 20 hr, at least about 22 hr or at least about 24 hr. In some embodiments, said administration of glycopyrrolate and formoterol results in a reduction of one or more side effects associated with glycopyrrolate, formoterol or both.

In some embodiments, the methods provided herein result in reduced side effects, which may include at least one or more of the following: airway hyperreactivity (hypersensitivity), angina, anorexia, anxiety, backaches, blurred vision, bradycardia, central stimulation, chest discomfort (e.g. chest pain), coughing, diarrhea, dizziness, drowsiness, drying or irritation of the oropharynx (such as dry mouth (xerostomia)), dyspnea, excitement, fatigue, flushing, hand tremors, headache, hoarseness, hypotension and palpitations, impotence, increased heart rate, insomnia, mental confusion, muscle cramps, muscle tremors, nausea, nervousness, palpitations, sweating, tachycardia, unusual taste, urinary hesitancy and retention, vertigo, vomiting, weakness, and wheezing.

In some embodiments, administration of the active ingredients permit reduction in the dose of LABA (e.g. formoterol, salmeterol, indacaterol, etc.), LAMA (e.g. glycopyrrolate, ipratropium, etc.) or both is less than about 75% of the standard dose. In some embodiments, the dose of formoterol, glycopyrrolate or both is less than about 65%, 60%, 55%, 50%, 45%, 40%, 35%, 30%, 25%, 20% or 15% o of the standard dose.

In some embodiments, the combination has a fill volume of about 0.5 mL or less. In some embodiments, the combination is administered in significantly less than about 3 min. In some embodiments, administration of the combination produces a duration of therapeutic effect of at least about 20 hr, at least about 22 hr or at least about 24 hr. In some embodiments, administration of the combination produces an increased magnitude of therapeutic effect. In some embodiments, the combination contains about 0.25 μg to about 6 μg of R,R-formoterol or about 0.5 μg to about 8 μg of racemic formoterol. Some embodiments provide a system or device adapted or adaptable to carry out the method of treatment. Some embodiments provide a unit dose, which may be used in one of the foregoing methods, comprising an effective amount of a muscarinic antagonist and a LABA in a pharmaceutically acceptable diluent. In some embodiments such unit dose may be contained in a kit comprising at least one additional dose.

Methods and Systems for the Treatment of Respiratory Conditions with HENs

The present invention provides methods and inhalation systems for treatment or prophylaxis of a respiratory condition in a patient, such as chronic obstructive pulmonary disease (COPD), and optionally chronic bronchitis and/or emphysema. In some embodiments, the methods and inhalation systems comprise administering to a patient a nominal dose of an active pharmaceutical ingredient (API), e.g. a LABA or a muscarinic antagonist in combination with a LABA, in an aqueous inhalation solution with a high efficiency nebulizer inhalation device, wherein delivering the nominal dose of the LABA or a muscarinic antagonist in combination with a LABA to the patient with a high efficiency nebulizer provides one or more of the following advantages: (1) an enhanced pharmacokinetic profile as compared to administration with a conventional nebulizer; (2) an enhanced therapeutic effect as compared to administration with a conventional nebulizer; (3) an enhanced lung deposition evidenced by scintigraphy or deconvolution, or derived from suitable in vitro indicators such as enhanced RDDR, RF, GSD, and/or a MMAD values as compared to administration with a conventional nebulizer; (4) reduced administration times, periods, and/or volumes; (5) a reduction in adverse side effects associated with API treatment and optionally a longer duration of therapeutic effect; optional administration with muscarinic antagonist and optionally a corticosteroid; or (6) an enhanced method of treatment of acute exacerbations of a respiratory condition in a patient, e.g. COPD.

Inhalation Therapy

An inhalation device, as used herein, refers to any device that is capable of administering a solution to the respiratory airways of a patient. Inhalation devices include conventional inhalation devices, such as metered dose inhalers (MDIs), conventional nebulizers, such as jet nebulizers, and high efficiency nebulizers, such as vibrating membrane nebulizers.

Inhalation nebulizers, or atomizers, are also commonly used for the treatment of COPD and other respiratory diseases Inhalation nebulizers deliver therapeutically effective amounts of pharmaceuticals by forming an aerosol which includes droplet sizes that can easily be inhaled. The aerosol can be used, for example, by a patient within the bounds of an inhalation therapy, whereby the therapeutically effective pharmaceutical or drug reaches the patient's respiratory tract upon inhalation. Some embodiments described herein provide for administration of a LABA or a combination of a muscarinic antagonist (e.g. glycopyrrolate) and a LABA (e.g. formoterol or salmeterol) with an inhalation device.

High Efficiency Nebulizer Inhalation Devices

High efficiency nebulizers are inhalation devices that are adapted to deliver a large fraction of a loaded dose to a patient. Some high efficiency nebulizers utilize microperforated membranes. In some embodiments, the high efficiency nebulizer also utilizes one or more actively or passively vibrating microperforated membranes. In some embodiments, the high efficiency nebulizer contains one or more oscillating membranes. In some embodiments, the high efficiency nebulizer contains a vibrating mesh or plate with multiple apertures and optionally a vibration generator with an aerosol mixing chamber. In some such embodiments, the mixing chamber functions to collect (or stage) the aerosol from the aerosol generator. In some embodiments, an inhalation valve is also used to allow an inflow of ambient air into the mixing chamber during an inhalation phase and is closed to prevent escape of the aerosol from the mixing chamber during an exhalation phase. In some such embodiments, the exhalation valve is arranged at a mouthpiece which is removably mounted at the mixing chamber and through which the patient inhales the aerosol from the mixing chamber. In some embodiments, the high efficiency nebulizer contains a pulsating membrane. In some embodiments, the high efficiency nebulizer is continuously operating. In some embodiments the high efficiency nebulizer is breath activated.

In some embodiments, the high efficiency nebulizer contains a vibrating microperforated membrane of tapered nozzles against a bulk liquid, and will generate a plume of droplets without the need for compressed gas. In these embodiments, a solution in the microperforated membrane nebulizer is in contact with a membrane, the opposite side of which is open to the air. The membrane is perforated by a large number of nozzle orifices of an atomizing head. An aerosol is created when alternating acoustic pressure in the solution is built up in the vicinity of the membrane causing the fluid on the liquid side of the membrane to be emitted through the nozzles as uniformly sized droplets.

Some embodiments of high efficiency nebulizers use passive nozzle membranes and separate piezoelectric transducers that are in contact with the solution. Another type of high efficiency nebulizer employs an active nozzle membrane, which uses the acoustic pressure in the nebulizer to generate very fine droplets of solution via the high frequency vibration of the nozzle membrane.

Some high efficiency nebulizers contain a resonant system. In some such high efficiency nebulizers, the membrane is driven by a frequency for which the amplitude of the vibrational movement at the center of the membrane is particularly large, resulting in a focused acoustic pressure in the vicinity of the nozzle; the resonant frequency may be about 100 kHz. A flexible mounting is used to keep unwanted loss of vibrational energy to the mechanical surroundings of the atomizing head to a minimum. In some embodiments, the vibrating membrane of the high efficiency nebulizer may be made of a nickel-palladium alloy by electroforming.

In some embodiments, the high efficiency nebulizer achieves lung deposition of at least about 30%, at least about 35%, at least about 40%, at least about 45%, at least about 50%, at least about 55%, at least about 60%, about 30% to about 60%, about 30% to about 55%, about 30% to about 50%, about 30% to about 40%, about 30% to about 90%, about 40% to about 80%, about 50% to about 60%, or about 60% to about 70%, based on the nominal dose of the LABA or muscarinic antagonist (e.g. LAMA) in combination with a LABA administered to the patient.

In some embodiments, the high efficiency nebulizer provides LABA lung deposition of at least about 15%, at least about 20%, at least about 25%, at least about 30%, at least about 35%, at least about 40%, at least about 45%, at least about 50%, at least about 55%, at least about 60%, about 20% to about 40%, about 25% to about 35%, about 25 to about 30%, about 35% to about 90%, about 40% to about 80%, about 50% to about 60%, or about 60% to about 70% based on the nominal dose of the LABA. In some embodiments, the high efficiency nebulizer provides for one or more of (a) or (b); and one or more of (i), (ii) or (iii): (a) a respirable dose delivery rate (RDDR) of at least about 100 μg/min or at least about 100 μg/min to at least about 5,000 μg/min; (b) an output rate of LABA of at least about 120 μg/min, at least about 150 μg/min, at least about 200 μg/min or at least about 200 μg/min to at least about 5,000 μg/min; (i) a respirable fraction (RF) of LABA of at least about 30%, at least about 35%, at least about 40%, at least about 45%, at least about 50%, at least about 55%, at least about 65% to at least about 75% or at least about 75% to at least about 85% respirable fraction upon administration; (ii) a Geometric Standard Deviation (GSD) of emitted droplet size distribution of the solution administered with a inhalation device of about 1.1 to about 2.1, about 1.2 to about 2.0, about 1.3 to about 1.9, less than about 2.2, about 1.4 to about 1.8, about 1.5 to about 1.7, about 1.4, about 1.5, or about 1.6; or (iii) a Mass Median Aerodynamic Diameter (MMAD) of droplet size of the solution emitted with the inhalation device of about 1 μm to about 5 μm, about 2 to about 4 μm, or about 3.5 to about 4.0 μm.

Additional features of a high efficiency nebulizer with perforated membranes are disclosed in U.S. Pat. Nos. 6,962,151, 5,152,456, 5,261,601, and 5,518,179, each of which is hereby incorporated by reference in its entirety. Some embodiments of the high efficiency nebulizer contain oscillating membranes. Features of these high efficiency nebulizers are disclosed in U.S. Pat. Nos. 7,252,085; 7,059,320; 6,983,747, each of which is hereby incorporated by reference in its entirety.

Commercial high efficiency nebulizers are available from: PAR1 (Germany) under the trade name eFlow®; Nektar Therapeutics (San Carlos, Calif.) (now Aerogen, Ltd.) under the trade names AeroNeb® Go and AeroNeb® Pro, and AeroNeb® Solo, Respironics (Murrysville, Calif.) under the trade names 1-Neb®, Omron (Bannockburn, Ill.) under the trade name Micro-Air®, and Activaero (Germany) under the trade name Akita®. Commercial high efficiency nebulizers are also available from Aerogen (Galaway, Ireland) utilizing the OnQ® nebulizer technology.

Conventional Nebulizers

In some embodiments, a LABA or a combination of a muscarinic antagonist and a LABA may be administered with a conventional nebulizer. Conventional nebulizers include, for example jet nebulizers or ultrasonic nebulizers. Conventional nebulizers generally utilize compressors to generate compressed air, which breaks the liquid medication into small breathable droplets, which form an aerosolized (atomized) mist. In some of these embodiments, when the patient breathes in, a valve at the top opens, which then allows air into the apparatus, thereby speeding up the mist generation; when the patient breathes out, the top valve closes, thereby slowing down the mist generation while simultaneously permitting the patient to breathe out through the opening of a mouthpiece flap.

In general, conventional nebulizers are characterized by relatively low efficiency in delivery of a API to lung tissue. Thus, a conventional nebulizer, such as a jet nebulizer, will be generally characterized by a respirable dose of less than 20% of the nominal dose. In some cases, the respirable dose is also referred to as the inhaled mass, which in any case is less than 20% of the nominal dose.

Some conventional nebulizers are disclosed in U.S. Pat. Nos. 6,513,727, 6,513,519, 6,176,237, 6,085,741, 6,000,394, 5,957,389, 5,740,966, 5,549,102, 5,461,695, 5,458,136, 5,312,046, 5,309,900, 5,280,784, and 4,496,086, each of which is hereby incorporated by reference in its entirety.

Commercial conventional nebulizers are available from: PAR1 (Germany) under the trade names PARI LC® and PARI-Jet®; A & H Products, Inc. (Tulsa, Okla.) under the trade name AquaTower®; Hudson RCI (Temecula, Calif.) under the trade name AVA-NEB®; Intersurgical, Inc. (Liverpool, N.Y.) under the trade name Cirrus®; Salter Labs (Arvin, Calif.) under the trade name Salter 8900®; Respironics (Murrysville, Pa.) under the trade name Sidestream®; Bunnell (Salt Lake City, Utah) under the trade name Whisper Jett; Smiths-Medical (Hyth Kent, UK) under the trade name Downdraft®.

Active Ingredient(s)

Muscarinic Antagonists

Acetylcholine released from cholinergic neurons in the peripheral and central nervous systems affects many different biological processes through interaction with two major classes of acetylcholine receptors: the nicotinic and the muscarinic receptors.

Muscarinic acetylcholine receptors are widely distributed in vertebrate organs where they mediate many vital functions. Three subtypes of muscarinic acetylcholine receptors have been identified as important in the lung, M1, M2, and M3, each with its unique pharmacological properties and a product of a distinct gene. These three subtypes are also located in organs other than the lung.

In the lung, M3 muscarinic receptors mediate smooth muscle contraction. Stimulation of M3 muscarinic receptors activate the enzyme phospholipase C via binding of the stimulatory G protein Gq/11 (Gs), leading to liberation of phosphatidyl inositol-4,5-bisphosphate, resulting in phosphorylation of contractile proteins and bronchial constriction. M3 muscarinic receptors are also found on pulmonary submucosal glands. Stimulation of this population of M3 muscarinic receptors results in mucus secretion. M2 muscarinic receptors make up approximately 50-80% of the cholinergic receptor population on airway smooth muscles. Under normal physiological conditions, M2 muscarinic receptors provide tight control of acetylcholine release from parasympathetic nerves. M1 muscarinic receptors are found in the pulmonary parasympathetic ganglia where they function to enhance neurotransmission.

Muscarinic acetylcholine receptor dysfunction in the lungs has been noted in a variety of different pathophysiological states. In asthma and COPD patients, inflammatory conditions lead to loss of inhibitory M2 and M3 muscarinic acetylcholine autoreceptor function on parasympathetic nerves supplying the pulmonary smooth muscle, causing an increased release of acetylcholine. This dysfunction in muscarinic receptors results in airway hyperreactivity and hyperresponsiveness.

Muscarinic acetylcholine receptor antagonist agents, or muscarinic antagonists, have the ability to inhibit the action of the neurotransmitter acetylcholine by blocking its interaction with muscarinic cholinergic receptors in general, and its interaction with specific muscarinic receptor subtypes in particular. Muscarinic antagonists thereby prevent the effects resulting from the passage of unnecessary impulses through the parasympathetic nerves mediated by increased stimulation in patients with dysfunctional receptors, resulting in, among other physiological effects, relaxation of smooth muscles in the lung.

Aclidinium,

((3R-3-{[hydroxydi(thiophen-2-yl)acetyl]oxy}-1-(3-phenoxypropyl)-1-azoniabicyclo[2.2.2]octane bromide), is a specific long-acting muscarinic receptor antagonist. Aclidinium is in development for use as an anticholinergic agent. Clinically, aclidinium has been tested in a dry powder inhaled format.

In some embodiments of the present invention, the muscarinic antagonist is aclidinium and is administered at a nominal dosage of 100 μg/dose to about 5 mg/dose, about 50 μg/dose to about 2 mg/dose or about 50 μg/dose to about 1 mg per dose. In some embodiments, aclidinium is given in 100 μg, 200 μg, 300 μg, 400 μg, 500 μg, 600 μg, 700 μg, 800 μg, 900 μg, or 1,000 μg doses.

The process of making aclidinium is known by a person of ordinary skill in the art. Aclidinium can be made by a number of known methods including those described in U.S. Pat. No. 6,750,226, which is incorporated herein by reference in its entirety, and which sets forth several structurally related muscarinic antagonists. Additional examples of muscarinic antagonists are set forth in U.S. Pat. Nos. 7,312,231 and 7,208,501, each of which is incorporated herein by reference in its entirety.

Trospium

(endo-3-[(Hydroxydiphenylacetyl)oxy]spiro[8-azoniabicyclo[3.2.1]ocatane-8,1′-pyrrolidinium]chloride benzilate), is a specific long-acting muscarinic receptor antagonist. Trospium has been known for many years to be an effective anticholinergic agent. Clinically, trospium has been used in several indications and been delivered by a number of different routes. Currently, trospium is used as a urinary antispasmotic and is sold under the brand name Sanctura®.

In some embodiments of the present invention, the muscarinic antagonist is trospium and is administered at a nominal dosage of 10 μg/dose to about 5 mg/dose, about 10 μg/dose to about 2 mg/dose or about 50 μg/dose to about 1 mg per dose. In some embodiments, trospium is given in 10 μg, 50 μg, 100 μg, 200 μg, 300 μg, 400 μg, 500 μg, 600 μg, 700 μg, 800 μg, 900 μg, or 1,000 μg doses.

The process of making trospium is known by a person of ordinary skill in the art. Trospium can be made by a number of known methods including those described in U.S. Pat. No. 3,480,626, which is incorporated herein by reference in its entirety.

Glycopyrrolate,

3-[(cyclopentylhydroxyphenylacetyl)oxy]-1,1-dimethylpyrrolidinium, is a specific long-acting muscarinic receptor antagonist. Glycopyrrolate has been known for many years to be an effective anticholinergic agent. Clinically, glycopyrrolate has been used in several indications and been delivered by a number of different routes. Currently, glycopyrrolate is used as an injectable compound to reduce gastric acid secretions during anesthesia and also as an oral product for treating gastric ulcers.

In some embodiments of the present invention, the muscarinic antagonist is glycopyrrolate and is administered at a nominal dosage of 100 μg/dose to about 5 mg/dose, about 200 μg/dose to about 2 mg/dose or about 250 μg/dose to about 1 mg per dose.

The process of making glycopyrrolate is known by a person of ordinary skill in the art. Glycopyrrolate can be made as follows. First, alpha-phenylcyclopentaneglycolic acid is esterified by refluxing with methanol in the presence of hydrochloric acid and the resulting ester is transesterified with 1-methyl-3-pyrrolidinol using sodium as a catalyst; the transester is then reacted with methyl bromide to give glycopyrrolate. U.S. Pat. No. 6,433,003, which describes this process in more detail, is hereby incorporated by reference in its entirety.

Glycopyrrolate for injectable and oral administration is readily commercially available. Injectable glycopyrrolate in commercial administrations are sold by: Baxter Healthcare, Inc. (Deerfiled, Ill.) under the trade name Robinul and by Luitpold Pharmaceuticals, Inc. (Shirley, N.Y.) under the generic name glycopyrrolate. Oral glycopyrrolate is commercially available under the generic name glycopyrrolate from Corepharma, LLC (Middlesex, N.J.) and Kali Laboratories, Inc. (Somerset, N.J.), and is available from Sciele Pharma, Inc. (Atlanta, Ga.) under the trade names Robinul and Robinul Forte.

Muscarinic antagonists can be long-acting or short-acting. Long-acting muscarinic antagonists have a therapeutic effect lasting greater than about 6 hours. Short-acting muscarinic antagonists have a duration of therapeutic effect of less than about 6 hours. Long-acting muscarinic antagonists include, but are not limited to, glycopyrrolate, tiotropium, aclidinium, trospium, QAT370, GSK233705, GSK656398, BEA 2180, or a pharmaceutical acceptable derivative, salt, enantiomer, diastereomer, or racemic mixtures thereof.

Short-acting muscarinic antagonists include, but are not limited to ipratropium, oxitropium or a pharmaceutical acceptable derivative, salt, enantiomer, diastereomer, or racemic mixtures thereof.

In some embodiments, the muscarinic antagonist is glycopyrrolate, tiotropium, aclidinium, trospium, QAT370, GSK233705, GSK 656398, BEA2180, ipratropium, oxitropium, oxybutynin or a pharmaceutical acceptable derivative, salt, enantiomer, diastereomer, or a pharmaceutical acceptable derivative, salt, enantiomer, diastereomer, or racemic mixture thereof.

Beta 2-Agonists

The stimulation of beta 2-adrenergic receptors stimulates adenylate cyclase, resulting in an increased level of the second messenger cAMP that in turn leads to decreased intracellular calcium concentration and consequently smooth muscle relaxation. Stimulation of certain beta 2-adrenergic receptors in particular causes hydrolysis of polyphosphoinositides and mobilization of intracellular calcium which results in a variety of calcium mediated responses such as smooth muscle contraction. Consequently, inhibition of this receptor activation prevents the intracellular calcium increase and leads to smooth muscle relaxation.

Beta 2-agonists (i.e. beta 2-adrenoreceptor agonists) can be long-acting or short-acting. Long-acting beta 2-agonists (LABAs) have a therapeutic effect lasting greater than about 6 hours. Short-acting beta 2-agonists (SABAs) have a duration of therapeutic effect of less than about 6 hours.

Compounds having beta 2-agonist activity with a long-acting or short-acting effect have been developed to treat respiratory conditions. Such compounds include, but are not limited to, albuterol; bambuterol; bitolterol; broxaterol; carbuterol; clenbuterol; ibuterol; sulfonterol; isoproterenol; trimetoquinol; formoterol; desformoterol; hexoprenaline; ibuterol; indacaterol; isoetharine; isoprenaline; isoproterenol; levalbuterol; metaproterenol; picumeterol; pirbuterol; procaterol; reproterol; rimiterol; salbutamol; salmeterol; sulfonterol; terbutaline; trimetoquinol; tulobuterol; and TA-2005 (8-hydroxy-5-((1R)-1-hydroxy-2-(N-((1R)-2-(4-methoxyphenyl)-1-methylethyl)amino)ethyl)-carbostyril hydrochloride); or a or a pharmaceutical acceptable derivative, salt, enantiomer, diastereomer, or racemic mixtures thereof.

Formoterol is a long-acting beta 2-agonist compound. The process of making formoterol is known by one of skill in the art. Formoterol is derived from adrenaline and is used as a beta 2-agonist in inhalation therapy of respiratory diseases. Formoterol has been formulated as a dry powder and administered via devices such as the Turbuhaler® and the Aerolizer®.

Formoterol is also available as a tablet and a dry syrup in certain areas of the world (e.g., Atock®, marketed by Yamanouchi Pharmaceutical Co. Ltd., Japan). Formoterol administrations are also available in other areas (e.g., Europe and U.S.) for propellant-based metered dose inhalers and dry powder inhalers (e.g., Turbuhaler®, Aerolizer® and Foradil Aerolizer®). None of these administrations are water based solutions. In some embodiments, the nebulized solution is a solution of formoterol and is delivered as a nominal dose of about 0.25 μg to about 20 μg per dose, about 0.25 μg to about 15 μg per dose, 0.25 μg to about 10 μg per dose, 0.25 μg to about 8 μg per dose, 0.25 μg to about 6 μg per dose, 0.25 μg to about 6 μg per dose, 0.25 μg to about 4 μg per dose, 0.25 μg to about 2 μg per dose, 0.5 μg to about 20 μg per dose, about 0.5 μg to about 15 μg per dose, about 0.5 μg to about 10 μg per dose, about 0.5 μg to about 8 μg per dose, about 0.5 μg to about 6 μg per dose, about 0.5 μg to about 6 μg per dose, about 0.5 μg to about 4 μg per dose, about 0.5 μg to about 2 μg per dose, about 1 μg to about 20 μg per dose, about 1 μg to about 15 μg per dose, about 1 μg to about 10 μg per dose, about 1 μg to about 8 μg per dose, about 1 μg to about 6 μg per dose, about 1 μg to about 6 μg per dose, about 1 μg to about 4 μg per dose or about 1 μg to about 2 μg per dose. In some embodiments, the nebulized solution is a solution of arformoterol and is delivered as a nominal dose of about 0.25 μg to about 30 μg per dose, about 0.25 μg to about 25 μg per dose, 0.25 μg to about 15 μg per dose, 0.25 μg to about 8 μg per dose, about 0.25 μg to about 5 μg per dose, about 0.25 μg to about 4 μg per dose, 0.25 μg to about 3 μg per dose, 0.25 μg to about 2 μg per dose, 0.25 μg to about 1 μg per dose, about 0.5 μg to about 30 μg per dose, about 0.5 μg to about 25 μg per dose, 0.5 μg to about 15 μg per dose, 0.5 μg to about 8 μg per dose, about 0.5 μg to about 5 μg per dose, about 0.5 μg to about 4 μg per dose, 0.5 μg to about 3 μg per dose, 0.5 μg to about 2 μg per dose, 0.5 μg to about 1 μg per dose, about 0.8 μg to about 30 μg per dose, about 0.8 μg to about 25 μg per dose, 0.8 μg to about 15 μg per dose, 0.8 μg to about 8 μg per dose, about 0.8 μg to about 5 μg per dose, about 0.8 μg to about 4 μg per dose, 0.8 μg to about 3 μg per dose, 0.8 μg to about 2 μg per dose, 0.8 μg to about 1 μg per dose, about 1 μg to about 30 μg per dose, about 1 μg to about 25 μg per dose, 1 μg to about 15 μg per dose, 1 μg to about 8 μg per dose, about 1 μg to about 5 μg per dose, about 1 μg to about 4 μg per dose, 1 μg to about 3 μg per dose, 1 μg to about 2 μg per dose, about 2 μg to about 30 μg per dose, about 2 μg to about 25 μg per dose, 2 μg to about 15 μg per dose, 2 μg to about 8 μg per dose, about 2 μg to about 5 μg per dose, about 2 μg to about 4 μg per dose or about 2 μg to about 3 μg per dose.

Commercial administrations of arformoterol tartrate (R,R-formoterol) are sold by Sepracor, Inc. (Marlborough, Mass.) under the trade name Brovana®. Formoterol fumarate is sold by several companies including AstraZeneca, Inc. (London, England) under the trade name Oxis®, Novartis International AG (Basel, Switzerland) under the trade names Foradil® and Certihaler®, and Dey, L.P. (Napa, Calif.) under the trade name Perforomist®. As used herein, “formoterol” (unless further qualified) refers generically to all forms of formoterol, such as arformoterol, racemic formoterol (mixture of R, R-formoterol and S,S-foroterol), or a pharmaceutically acceptable salt thereof “Arformoterol” refers to enantiomerically pure (at least 90%) R,R-formoterol. “Racemic formoterol” (or formoterol racemate) refers to an approximately 50:50 mixture of R,R-formoterol and S,S-formoterol.

Salmeterol is a long-acting beta 2-agonist compound. The process for making salmeterol is known by a person of ordinary skill in the art and is described in U.S. Pat. No. 4,992,474, which is hereby incorporated by reference. Commercial administrations of salmeterol are sold by GlaxoSmithKline, Inc. (Triangle Park, N.C.) under the trade names Advair® and Serevent®. In some embodiments, the nebulized LABA is salmeterol and is administered as a nominal dose of about 1 μg to about 200 μg per dose, about 1 μg to about 150 μg per dose, about 1 μg to about 100 μg per dose, about 1 μg to about 50 μg per dose, about 1 μg to about 35 μg per dose, about 1 μg to about 30 μg per dose, about 1 μg to about 25 μg per dose, about 1 μg to about 20 μg per dose, about 1 μg to about 15 μg per dose, about 1 μg to about 10 μg per dose, about 5 μg to about 200 μg per dose, about 5 μg to about 150 μg per dose, about 5 μg to about 100 μg per dose, about 5 μg to about 50 μg per dose, about 5 μg to about 35 μg per dose, about 5 μg to about 30 μg per dose, about 5 μg to about 25 μg per dose, about 5 μg to about 20 μg per dose, about 5 μg to about 15 μg per dose, about 5 μg to about 10 μg per dose, about 10 μg to about 200 μg per dose, about 10 μg to about 150 μg per dose, about 10 μg to about 100 μg per dose, about 10 μg to about 50 μg per dose, about 10 μg to about 35 μg per dose, about 10 μg to about 30 μg per dose, about 10 μg to about 25 μg per dose, about 10 μg to about 20 μg per dose, about 10 μg to about 15 μg per dose, about 20 μg to about 200 μg per dose, about 20 μg to about 150 μg per dose, about 20 μg to about 100 μg per dose, about 20 μg to about 50 μg per dose, about 10 μg to about 45 μg per dose, about 10 μg to about 40 μg per dose, about 10 μg to about 35 μg per dose, about 10 μg to about 30 μg per dose, about 10 μg to about 25 μg per dose, about 10 μg to about 20 μg per dose or about 10 μg to about 15 μg per dose. In some embodiments, the LABA is R-salmeterol administered within one of the immediately foregoing ranges set forth for salmeterol.

Unless otherwise specified herein “formoterol” refers to racemic formoterol (mixture of R,R-formoterol and S,S-formoterol), enantiomerically pure R,R-formoterol (arformoterol), or a pharmaceutically acceptable salt thereof.

Inhalation Solutions

The present invention relates to methods and inhalation systems for the use of inhalation solutions in an inhalation device for the treatment or prophylaxis of a respiratory condition in a patient, such as COPD, chronic bronchitis, or emphysema. In some embodiments, the methods and inhalation systems comprise administering to the patient a nominal dose of one or more API, for example a LABA or a muscarinic antagonist in combination with a LABA, in an aqueous inhalation solution with an inhalation device, e.g. a high efficiency nebulizer or a conventional nebulizer a high efficiency nebulizer, conventional nebulizer, and optionally a conventional inhalation device.

In some embodiments, the aqueous inhalation solution is administered with an inhalation device, e.g. high efficiency nebulizer, at a fill volume of 0.5 mL or less, at least about 0.5 mL to about 1.5 mL, at least about 0.25 mL or less, at least about 0.5 mL to about 1.5 mL, at least about 1.5 mL, or at least about 2.0 mL. In some embodiments, the aqueous inhalation solution is administered with an inhalation device, e.g. high efficiency nebulizer, at a fill volume of at least about 0.25 mL to about 2.0 mL, about 0.5 mL to about 1.5 mL, about 0.5 mL to about 1.0 mL, about 0.5 mL or less, about 1 mL or less, about 1.5 mL or less, or about 2.0 mL or less. In some embodiments, the aqueous inhalation solution is administered with an inhalation device, e.g. a high efficiency nebulizer, which provides for a residual volume of a muscarinic antagonist in combination with a LABA after administration of the muscarinic antagonist in combination with a LABA of less than about 10%, less than about 5%, or less than about 3% of the nominal dose.

In some embodiments, the aqueous inhalation solution is administered in about 0.25 to about 10 minutes, about 0.50 to about 8 minutes, less than about 8, less than about 7, less than about 6, less than about 5, less than about 4, less than about 3, less than about 2, or less than about 1.5 minutes. In some embodiments, the aqueous inhalation solution is administered in about 3 minutes or less.

In some embodiments, the nominal dose administered with the high efficiency nebulizer is a LABA or a muscarinic antagonist in combination with a LABA that is substantially free of preservative, such as benzyl alcohol. In some embodiments, the nominal dose of LABA or muscarinic antagonist (e.g. LAMA) in combination with a LABA is in an inhalation solution that further comprises at least one excipient or active adjunct. In some embodiments, the excipient or adjunct is a member of the group consisting of organic acid (such as a low molecular weight organic acid like citric acid or ascorbic acid), an antioxidant (such as EDTA), an osmolarity adjusting agent (such as a salt like sodium chloride) or a pH buffer.

In some embodiments, the inhalation solution comprising the LABA or muscarinic antagonist (e.g. LAMA) in combination with a LABA further comprises a corticosteroid, such as fluticasone, mometasone, beclomethasone, triamcinolone, fluniolide, ciclesonide, or budesonide. In some embodiments, the inhalation solution further comprises an excipient or active adjunct. Examples of excipients and active adjuncts include an organic acid (e.g. citric acid, ascorbic acid or a combination of both), pilocarpine, cevimeline or carboxymethylcellulose, or a mucolytic compound.

High Concentration Inhalation Solutions

In some embodiments, the aqueous inhalation solution administered with an inhalation device, e.g. a metered dose inhaler (MDI), conventional nebulizer, or high efficiency nebulizer, contains a high concentration of muscarinic antagonist and LABA. The high concentration of muscarinic antagonist and LABA provides certain advantages as compared to a lower concentration solution. For example, in some embodiments, a high concentration solution may be administered less frequently than a low concentration solution. While not wishing to be bound by theory, it is considered that the high concentration solution allows for gradual uptake of the muscarinic antagonist, which provides a longer duration of action than the lower concentration solution.

In some embodiments, the high concentration aqueous inhalation solution of API, for example glycopyrrolate, results in a dosing regimen aimed at achieving once-a-day dosing. In some embodiments, the methods and systems employ a high concentration aqueous inhalation solution of muscarinic antagonist, for example glycopyrrolate, containing at least about 0.25 mg/mL to about 50 mg/mL, about 0.25 mg/mL to about 20 mg/mL, about 0.25 mg/mL to about 10 mg/mL, about 0.5 mg/mL to about 50 mg/mL, about 0.5 mg/mL to about 20 mg/mL, about 0.5 mg/mL to about 10 mg/mL, at least about 0.5 mg/mL, at least about 1.0 mg/mL, or at least about 1.5 mg/mL, at least about 2.0 mg/mL, at least about 5 mg/mL, at least about 10 mg/mL, at least about 20 mg/mL or at least about 25 mg/mL. In some embodiments, the concentration of glycopyrrolate is about 0.05 mg/mL to about 50 mg/mL, about 0.05 mg/mL to about 20 mg/mL, about 0.05 mg/mL to about 10 mg/mL, about 0.10 mg/mL to about 50 mg/mL, about 0.10 mg/mL to about 20 mg/mL, about 0.10 mg/mL to about 10 mg/mL, about 0.2 mg/mL to about 50 mg/mL, about 0.2 mg/mL to about 20 mg/mL, about 0.2 mg/mL to about 2 mg/mL.

In some embodiments, the muscarinic antagonist, for example glycopyrrolate, nominal dose of aqueous inhalation solution is about 0.05 mg to about 50 mg, about 0.05 mg to about 20 mg, about 0.05 mg to about 10 mg, about 0.05 mg to about 5 mg, about 0.05 mg to about 3 mg, 0.25 mg to about 50 mg, about 0.25 mg to about 20 mg, about 0.25 mg to about 10 mg, about 0.25 mg to about 5 mg, about 0.25 mg to about 3 mg, 0.2 mg to about 2 mg, about 0.25 mg to about 1.5 mg, about 0.25 to about 1 mg, at least about 0.25 mg, at least about 0.5 mg, at least about 1.0 mg, at least about 1.5 mg, or at least about 2.0 mg.

In some embodiments, the high concentration aqueous inhalation solution has a fill volume of about 0.5 mL to about 1.5 mL, about 0.5 mL to about 1.0 mL, about 0.5 mL or less, about 1 mL or less, or about 1.5 mL. In some embodiments, the aqueous inhalation solution is administered in about 0.25 to about 10 minutes, about 0.50 to about 8 minutes, less than about 8, less than about 7, less than about 6, less than about 5, less than about 4, less than about 3, less than about 2, or less than about 1.5 minutes. In some embodiments, the aqueous inhalation solution is administered in about 3 minutes or less.

In some embodiments, the high concentration nominal dose of the muscarinic antagonist administered with an inhalation device provides for a greater duration of therapeutic effect compared to administration of a lower concentration or higher volume of substantially the same nominal dose of muscarinic antagonist. In some embodiments, the nominal dose of muscarinic antagonist administered with an inhalation device provides for a shorter time to onset of therapeutic effect compared to administration of a lower concentration or higher volume of substantially the same nominal dose of muscarinic antagonist. In some embodiments, the nominal dose of muscarinic antagonist administered with an inhalation device provides for a shorter time to maximum therapeutic effect compared to administration of a lower concentration or higher volume of substantially the same nominal dose of muscarinic antagonist.

Characterization of Inhalation Devices

The efficiency of a particular inhalation device can be measured by many different ways, including an analysis of pharmacokinetic properties, measurement of lung deposition percentage, measurement of respirable dose delivery rates (RDDR), a determination of output rates, respirable fraction (RF), geometric standard deviation values (GSD), and mass median aerodynamic diameter values (MMAD) among others.

A person skilled in the art is knowledgeable of methods and systems for examining a particular inhalation device. One such system consists of a computer and a hollow cylinder in a pump with a connecting piece to which an inhalation device is to be connected. In the pump there is a piston rod, which extends out of the hollow cylinder. A linear drive unit can be activated in such a manner that one or more breathing patterns will be simulated on the connecting piece of the pump. In order to be able to carry out the evaluation of the inhalation device, the computer is connected in an advantageous configuration with a data transmitter. With the aid of the data transmitter, the computer can be connected with another computer with specific data banks, in order to exchange the data of breathing patterns. In this manner, a breathing pattern library which is as representative as possible can be very rapidly formed. U.S. Pat. No. 6,106,479 discloses this method for examining an inhalation device in more detail, and is hereby incorporated by reference in its entirety.

Pharmacokinetic Profile

Pharmacokinetics is concerned with the uptake, distribution, metabolism and excretion of a drug substance. A pharmacokinetic profile comprises one or more biological measurements designed to measure the absorption, distribution, metabolism and excretion of a drug substance. One way of visualizing a pharmacokinetic profile is by means of a blood plasma concentration curve, which is a graph depicting mean active ingredient blood plasma concentration on the Y-axis and time (usually in hours) on the X-axis. Some pharmacokinetic parameters that may be visualized by means of a blood plasma concentration curve include:

AUCₗₐₛₜ: The area under the curve from time zero to time of last measurable concentration.

AUC_{(0-∞)}: The total area under the curve.

Cₘₐₓ: The maximum plasma concentration in a patient.

Tₘₐₓ: The time to reach maximum plasma concentration in a patient

An enhanced pharmacokinetic profile in a patient can be indicated by increased AUCₗₐₛₜ AUC_{(0-∞)}, Cₘₐₓ, or a decreased Tₘₐₓ. Enhanced levels of a pharmaceutical agent in the blood plasma of a patient may result in or more improved symptoms of an airway respiratory condition, e.g. COPD.

In some embodiments, a method or system described herein provides at least about a 1.5-, 1.8- or even a two-fold enhancement in pharmacokinetic profile, meaning that administration of an active pharmaceutical ingredient (“API”—a LABA or a muscarinic antagonist in combination with a LABA) with a high efficiency nebulizer provides at least about a two-fold increase in one or more of AUCₗₐₛₜ, AUC_{(0-∞)}, or Cₘₐₓ as compared to the same or lower nominal dose of API administered with a conventional nebulizer.

In some embodiments, a method or system described herein provides at least about a two-fold enhancement in pharmacokinetic profile, meaning that administration of an active pharmaceutical ingredient (“API”—e.g. a LABA or a muscarinic antagonist in combination with a LABA) with a high efficiency nebulizer provides a comparable AUCₗₐₛₜ, AUC_{(0-∞)}, or Cₘₐₓ as compared to the same or lower nominal dose of API administered with a conventional nebulizer.

Enhanced Therapeutic Effect

The assessment of therapeutic effect is known to those skilled in the art, such as pulmonologists trained to recognized the distinctions between various types of respiratory illnesses, including chronic obstructive pulmonary disease (“COPD”) and asthma. Assessment of efficacy may be carried out by various methods known to the person skilled in the art, and may include both objective and subjective (patient-generated) measures of efficacy. Objective measures of efficacy can be obtained inter alia by spirometry; and subjective measures of efficacy can be obtained for example by employing one or more patient symptom questionnaires or surveys. In some embodiments, the methods and systems herein are for treatment of COPD, and thus such embodiments are discussed in further detail below. It is considered that embodiments of the methods and symptoms described herein (including those employing administration of a LABA or a muscarinic antagonist in combination with a LABA, optionally with a high efficiency nebulizer or at a high concentration) will provide superior efficacy in treatment of COPD as compared to treatment with conventional methods (such as those in which muscarinic antagonist or LABA is administered as a monotherapy, with a conventional nebulizer and/or at a relatively low concentration).

COPD Efficacy Assessment

COPD is a progressive, chronic disease of the airways, characterized by chronic inflammation and destruction of the airways and lung parenchyma, resulting in airflow obstruction. Thus, efficacy in the treatment of COPD refers to the ability to restore airflow to the patient. In some cases, especially in older and immune-compromised patients, COPD can be further characterized by exacerbations—acute, often pathogen—or allergen-induced, degradation of airflow. There are several indicators (endpoints) of efficacy in the treatment of COPD. Some efficacy endpoints that are used in COPD studies are set forth below. It is considered that a muscarinic antagonist in combination with a LABA will demonstrate efficacy in one or more of these tests. In particular, it is considered that in some embodiments a nominal dose of a muscarinic antagonist in combination with a LABA, administered with a high efficiency nebulizer, will out-perform substantially the same or higher nominal dose of muscarinic antagonist in combination with a LABA administered with a conventional nebulizer, as determined by one or more of these endpoints. In some embodiments, it is considered that a combination of a muscarinic antagonist with a LABA will out-perform the muscarinic antagonist as monotherapy, and/or the LABA as a monotherapy, as determined by one or more of these endpoints.

Pulmonary Function Tests:

Pulmonary function testing by spirometry is a useful way to assess airflow obstruction and, therefore, is a useful way to assess the efficacy of COPD treatment as well as to compare the relative merits of different COPD treatments—e.g. administration of different dosages of active pharmaceutical ingredient (“API”), administration of substantially the same dosages of API with different delivery devices, or administration of different dosages of API with different delivery devices. Forced expiratory volume in one second (FEV₁) obtained from typical spirometry is commonly used as an efficacy endpoint because FEV₁ is a reflection of the extent of airway obstruction. Spirometry is also well-standardized, is easy to perform and provides consistent, reproducible results across different pulmonary function laboratories. Air-trapping and hyperinflation are common features in COPD, particularly in emphysematous-type, and are reflected in parameters of lung function testing, such as an elevation in the residual volume to total lung capacity ratio (RV/TLC). Hyperinflation is believed to be responsible, at least in part, for the sense of dyspnea.

Exercise Capacity:

Reduced capacity for exercise is a typical consequence of airflow obstruction in COPD patients, particularly because of dynamic hyperinflation occurring during exercise. Assessment of exercise capacity by treadmill or cycle ergometry combined with lung volume assessment is in some cases a tool to assess efficacy of a COPD drug. Alternative assessments of exercise capacity, such as the Six Minute Walk or Shuttle Walk, can also be used in some cases. The characteristics, including the limitations, of these tests will be known to those skilled in the art.

Outcome Measures can also be used, alone or preferably in combination with one or more objective tests, to determine efficacy of COPD therapy.

Symptom Scores:

Symptom scores determined by asking patients to evaluate specific symptoms on a categorical, visual or numerical scale can be a simple way to assess efficacy of a drug based on the patient's own assessment of health status. Symptom scores can be valuable for assessing efficacy of a drug specifically aimed at relieving a symptom. In clinical programs aimed at other aspects of COPD, patient-reported symptom scores can be useful in assessing secondary effects of the therapy and may provide important additional evidence of efficacy. The characteristics, including the limitations, of these tests will be known to those skilled in the art.

Activity Scales: Activity scales such as the Medical Research Council dyspnea score, the Borg Scale, and the Mahler Baseline Dyspnea Index/Transitional Dyspnea Index, can be used in some cases as supportive evidence of efficacy. These scales are relatively simple to administer. The characteristics, including the limitations, of these tests will be known to those skilled in the art.

Health-related, quality-of-life instruments: Health-related quality-of-life instruments, such as the St. George's Respiratory Questionnaire and the Chronic Respiratory Questionnaire, are designed to systematically assess many different aspects of the effect of COPD on a patient's life. These instruments can be used to assess efficacy of a drug. These instruments are multidimensional and assess various effects of the disease on a patient's life and health status. The characteristics, including the limitations, of these tests will be known to those skilled in the art.

Further information regarding testing drugs for efficacy in the treatment of COPD can be found in the United States Food and Drug Administration's guidance document entitled: “Guidance for Industry: Chronic Obstructive Pulmonary Disease: Developing Drugs for Treatment,” November, 2007, which is available from www.fda.gov/cder/guidance/index.htm.

A LABA or a muscarinic antagonist in combination with a LABA is said to have a therapeutic effect in the treatment of COPD when it causes an increase in one or more measures of pulmonary function to a predetermined percentage above baseline. In some embodiments, the predetermined percentage above baseline is about 5%, about 10%, about 15%, about 20%, or about 25%. In some specific embodiments, a LABA or a muscarinic antagonist in combination with a LABA will be considered to have a therapeutic effect when it raises one or more of the above-mentioned spirometry measurements (e.g. FEV₁) at least about 15% above baseline. In some embodiments, the baseline is considered the spirometry measurement immediately prior to administration of the nebulized combination; in some embodiments, the baseline is considered the spirometry measurement obtained at substantially the same time of day upon administration of placebo.

Spirometry is the measurement of respiration, which is generally conducted by a physician with the aid of a spirometer. Spirometers measure inspired and expired airflow for the purpose of assessing pulmonary ventilatory function. Spirometry is the most common pulmonary function test measuring lung function. Typical spirometers display volume-time curves (showing volume on the Y-axis and time, usually in seconds, on the X-axis) and optionally flow-volume curves (showing rate of flow on the Y-axis and the total volume inspired/expired on the X-axis). U.S. Pat. No. 7,291,115 discloses a spirometer and method to measure the ventilatory function by spirometry, and is hereby incorporated by reference in its entirety. Methods of using a spirometer are familiar to those skilled in the art.

Relevant parameters measured by spirometers include:

FEV1 (or FEV₁): Forced Expiratory Volume in 1 Second, which is the maximum volume of air exhaled during the first second of maximum effort from a maximum inhalation. It is expressed in liters and in percentage of the patient's reference value from baseline. It becomes altered in cases of bronchial obstruction and it is fundamental for diagnosing and monitoring obstructive diseases, e.g. COPD.

Change in FEV1: Change in FEV1 may be calculated as the difference between the FEV1 value measured after dosing and the FEV1 measured immediately prior to dosing. Change in FEV1 may also be measured in reference to a placebo. These values may be expressed in absolute terms or in terms of percent change from baseline or placebo.

FEV1 AUC (or FEV₁ AUC): This is the area between the FEV1 measurements vs. time curve over a time course. In some embodiments, the time course is a predetermined period, such as 0-6 hr., 0-12 hr., 0-18 hr., 0-24 hr., 0-30 hr., or 0-36 hr.

Trough FEV1 (or Trough FEV₁): This is the FEV1 value measured just prior to administration of the drug. In some cases, the trough FEV1 is obtained in the morning, just prior to administration of the drug. In some embodiments, the change in trough FEV1 is the difference between the trough FEV1 for the drug and the trough FEV1 for a placebo, after a period of time. In some embodiments, the change in the trough FEV1 is measured over a predetermined time course, such as 1 wk, 2 wk, 4 wk or 12 wk.

FVC: Forced Vital Capacity, which is the maximal volume of air exhaled with maximal effort from a position of maximal inhalation. It is expressed in liters and in percentage of a patient's reference value from baseline.

FEV1/FVC: The quotient of FEV1 and FVC. Normal values of FEV1/FVC are greater than 0.75.

PEF: Peak Expiratory Flow, which is the highest expiratory flow achieved with maximal effort from a position of maximal inspiration. This is essentially the speed of the air moving out of the lungs of a patient at the beginning of expiration. It is expressed in liters/second or in liters/minute.

FEF₂₅₋₇₅: Forced Expiratory Flow from 25% to 75% on the flow-volume curve, which is the average flow (or speed) of air coming out of the lung during the middle portion of expiration.

FEF₂₅₋₅₀: Forced Expiratory Flow from 25% to 50% on the flow-volume curve, which is another measure of the average flow (or speed) of air coming out of the lung during the middle portion of expiration.

FIF₂₅₋₇₅: Forced Inspiratory Flow from 25% to 75% on the flow-volume curve, which is the average flow (or speed) of air entering the lung during the middle portion of inspiration.

FIF₂₅₋₅₀: Forced Inspiratory Flow from 25% to 75% on the flow-volume curve, which is another measure of the average flow (or speed) of air entering the lung during the middle portion of inspiration.

An enhanced therapeutic effect can include an increased magnitude of therapeutic effect, an enhanced duration of therapeutic effect, an enhanced time to onset of therapeutic effect, a shorter time to maximum therapeutic effect or a greater magnitude of therapeutic effect. In some embodiments described herein, an enhanced therapeutic effect relates to the increased ability of a pharmaceutical agent to relieve the symptoms of an airway respiratory disorder, e.g. COPD. Thus, an enhanced therapeutic effect may be determined by comparing values of change in FEV₁ (i.e. change in FEV₁ from baseline or compared to a placebo), % change in FEV₁ (i.e. percent change in FEV₁ from baseline or compared to placebo), FEV₁ AUC, trough FEV₁, FEV₁/FVC, PEF, FEF₂₅₋₇₅, FEF₂₅₋₅₀, FIF₂₅₋₇₅, FIF₂₅₋₅₀ obtained from a patient or patient population in one therapeutic milieu versus another anther therapeutic milieu. For example, an enhanced therapeutic effect may be determined by comparing FEV₁ values for a patient or patient population treated with a muscarinic antagonist administered with a high efficiency nebulizer against the same drug administered with a conventional nebulizer. In another example, an enhanced therapeutic effect may be determined by comparing FEV₁ values for a patient or patient population treated with a muscarinic antagonist administered at a high concentration against the same drug administered at a low concentration. In some cases, an enhanced therapeutic effect may be determined by comparing FEV₁ values for a patient or patient population treated with a muscarinic antagonist administered with a high efficiency nebulizer against a muscarinic antagonist alone administered with a conventional nebulizer. In another example, an enhanced therapeutic effect may be determined by comparing FEV₁ values for a patient or patient population treated with a muscarinic antagonist administered at a high concentration against a muscarinic antagonist alone administered at a low concentration. In some embodiments, the enhanced therapeutic effect is an increased magnitude of therapeutic effect. In some embodiments, the increased magnitude of therapeutic effect is an increase in the peak FEV₁ obtained with a high efficiency nebulizer versus the peak FEV₁ obtained with a conventional nebulizer. In some embodiments, the peak FEV₁ obtained with a high efficiency nebulizer is at least about 10%, 15%, 20%, or 30% above that obtained with a conventional nebulizer. In some embodiments, the peak FEV₁ obtained with a high efficiency nebulizer is at least about 25 mL, 50 mL, or 100 mL above that obtained with a conventional nebulizer. In some embodiments, the increased magnitude of therapeutic effect is an increase in the mean FEV₁ obtained with a high efficiency nebulizer versus the mean FEV₁ obtained with a conventional nebulizer. In some embodiments, the mean FEV₁ obtained with a high efficiency nebulizer is at least about 5%, 10%, or 15% above that obtained with a conventional nebulizer. In some embodiments, the mean FEV₁ obtained with a high efficiency nebulizer is at least about 50 mL, 100 mL, or 150 mL above that obtained with a conventional nebulizer. In some embodiments, the increased magnitude of therapeutic effect is an increase in the AUC for the FEV₁ versus time curve obtained with a high efficiency nebulizer versus the AUC for the FEV₁ versus time curve obtained with a conventional nebulizer. In some embodiments, the increase in AUC of the FEV₁ versus time curve obtained with a high efficiency nebulizer is at least about 50%, 75% or 100% above that obtained with a conventional nebulizer.

In some embodiments, the method or system (e.g. muscarinic antagonist, optionally in combination with a beta 2-agonist, administered at a high concentration and/or with a high efficiency nebulizer) provides an enhanced duration of therapeutic effect, as determined by the amount of time that a spirometric parameter (e.g. FEV₁, trough FEV₁) is above a predetermined threshold after therapy is administered. In some embodiments, the predetermined threshold is at least about 5% above baseline, at least about 10% above baseline, at least about 15% above baseline, at least about 20% above baseline, at least about 25% above baseline. In some specific embodiments, the threshold is about 15% above baseline. In some specific embodiments, the threshold is about 10% above baseline. In some embodiments, the threshold is 50 mL, 100 mL, 150 mL or more than about 150 mL above baseline. In some specific embodiments, the threshold is about 100 mL above baseline. Baseline can be determined by either a one-time reference to the spirometric parameter (e.g. FEV₁) immediately prior to administration of API, or by reference to the spirometric parameter level at several time periods during the study following administration of placebo to a predetermined set of patients. In some embodiments, baseline is determined based on the level of spirometric parameter (e.g. FEV₁) immediately prior to administration to the patient of muscarinic antagonist administered at a high concentration and/or with a high efficiency nebulizer. In some embodiments, baseline is determined by reference to the level of spirometric parameter (e.g. FEV₁) at several time periods (e.g., 12 hours, 24 hours) during evaluation of certain patients following placebo administration, with the simultaneous evaluation of other patients administered a muscarinic antagonist administered at a high concentration and/or with a high efficiency nebulizer.

In some embodiments, a duration of therapeutic effect is the period during which FEV₁ is at least about 5% above baseline, at least about 10% above baseline, at least about 15% above baseline, at least about 20% above baseline, at least about 25% above baseline. In some specific embodiments, the duration of therapeutic effect is the amount of time that the FEV₁ is at least 15% above baseline. In some specific embodiments, the duration of therapeutic effect is the amount of time that the FEV₁ is at least 10% above baseline. In some specific embodiments, the duration of therapeutic effect is the amount of time that the FEV₁ is at least 50 mL, 100 mL, or 150 mL above baseline. In some embodiments, a duration of therapeutic effect is the period during which FEV₁/FVC is at least about 5% above baseline, at least about 10% above baseline, at least about 15% above baseline, at least about 20% above baseline, at least about 25% above baseline. In some embodiments, the duration of therapeutic effect is the amount of time that the FEV₁/FVC is at least 15% above baseline. In some embodiments, a duration of therapeutic effect is the period during which PEF is at least about 5% above baseline, at least about 10% above baseline, at least about 15% above baseline, at least about 20% above baseline, at least about 25% above baseline. In some embodiments, the duration of therapeutic effect is the amount of time that the PEF is at least 15% above baseline. In some embodiments, a duration of therapeutic effect is the period during which FEF₂₅₋₇₅ is at least about 5% above baseline, at least about 10% above baseline, at least about 15% above baseline, at least about 20% above baseline, at least about 25% above baseline. In some embodiments, the duration of therapeutic effect is the amount of time that the FEF₂₅₋₇₅ is at least 15% above baseline. In some embodiments, a duration of therapeutic effect is the period during which FEF₂₅₋₅₀ is at least about 5% above baseline, at least about 10% above baseline, at least about 15% above baseline, at least about 20% above baseline, at least about 25% above baseline. In some embodiments, the duration of therapeutic effect is the amount of time that the FEF₂₅₋₅₀ is at least 15% above baseline. In some embodiments, a duration of therapeutic effect is the period during which FIF₂₅₋₇₅ is at least about 5% above baseline, at least about 10% above baseline, at least about 15% above baseline, at least about 20% above baseline, at least about 25% above baseline. In some embodiments, the duration of therapeutic effect is the amount of time that the FIF₂₅₋₇₅ is at least 15% above baseline. In some embodiments, a duration of therapeutic effect is the period during which FIF₂₅₋₅₀ is at least about 5% above baseline, at least about 10% above baseline, at least about 15% above baseline, at least about 20% above baseline, at least about 25% above baseline. In some embodiments, the duration of therapeutic effect is the amount of time that the FIF₂₅₋₅₀ is at least 15% above baseline.

A significantly greater, or greater, duration of therapeutic effect, indicates that the method or system (e.g. a high efficiency nebulizer-administered muscarinic antagonist) provides an increased period of time the spirometric parameter is above a predetermined threshold of about 5% above baseline, about 10% above baseline, about 15% above baseline, about 20% above baseline, about 25% above baseline, especially about 15% above baseline, for one or more of the spirometric parameters compared to the same spirometric parameter obtained with substantially the same nominal dose of drug administered with a different inhalation device, e.g. a conventional nebulizer. In some embodiments, the threshold for the spirometric parameter (e.g. FEV₁, or trough FEV₁) is 50 mL, 100 mL, 150 mL or more than about 150 mL above baseline. In some specific embodiments, the threshold is about 100 mL above baseline.

“About the same” duration of therapeutic effect means that the method or system (e.g. a high efficiency nebulizer-administered muscarinic antagonist, optionally in combination with a beta 2-agonist) provides substantially the same period of time that the spirometric parameter is above a predetermined threshold of about 5% above baseline, about 10% above baseline, about 15% above baseline, about 20% above baseline, about 25% above baseline, or especially about 15% above baseline, for one or more of the above spirometric parameters compared to the same spirometric parameter obtained with a substantially greater nominal dose of the muscarinic antagonist administered with a different inhalation device, e.g. conventional nebulizer (reference administration).

In some embodiments, an inhalation solution described herein (e.g. a LABA or a muscarinic antagonist (LAMA) in combination with a LABA inhalation solution administered with a conventional or high efficiency nebulizer) provides a duration of therapeutic effect of at least about 12 hr, about 12 hr to about 24 hr, about 18 hr to about 24 hr, about 20 hr to about 24 hr, or at least about 24 hr, in some embodiments the duration of therapeutic effect is at least about 12, 18, 20, 24, 28, 30, 32 or 36 hr.

In some embodiments in which the muscarinic antagonist combined with a LABA is administered with a high efficiency nebulizer, a reference condition is administration of substantially the same combination with a conventional nebulizer. In some embodiments, a reference condition for administration of a combination of muscarinic antagonist is administration of the muscarinic antagonist alone (same or higher dose), the LABA alone (same or higher dose) or the combination of muscarinic antagonist and LABA (one or both at a higher dose) with the same nebulizer.

A time to onset of therapeutic effect is the time for the spirometric parameter to reach a predetermined threshold of about 5% above baseline, about 10% above baseline, about 15% above baseline, about 20% above baseline, or about 25% above baseline, especially about 15% above baseline for one or more of the spirometric parameters of a LABA or a muscarinic antagonist in combination with a LABA administered with an inhalation device. An enhanced time to onset of therapeutic effect relates to the increased ability of a pharmaceutical agent to relieve the symptoms of an airway respiratory disorder, e.g. COPD. The enhanced time to onset of therapeutic effect may be a measure of the FEV₁, FEV₁/FVC, PEF, FEF₂₅₋₇₅, FEF₂₅₋₅₀, FIF₂₅₋₇₅, or FIF₂₅₋₅₀ levels.

A significantly shorter, or shorter, time to onset of therapeutic effect, in some embodiments, means that the method or system (a LABA or a muscarinic antagonist in combination with a LABA administered with a conventional or high efficiency nebulizer) provides for a shortened period of time for one or more spirometric parameters (e.g. FEV₁) to reach a predetermined threshold of about 5% above baseline, about 10% above baseline, about 15% above baseline, about 20% above baseline, or about 25% above baseline, especially about 15% above baseline, for one or more of the spirometric parameters compared to the same spirometric parameter(s) obtained with substantially the same nominal dose of the drug solution administered under a reference condition. In some embodiments, “about the same” time to onset of therapeutic effect means the method or system (e.g. administration of a LABA or a muscarinic antagonist in combination with a LABA with conventional or a high efficiency nebulizer) provides for substantially the same period of time for the spirometric parameter to reach a predetermined threshold of about 5% above baseline, about 10% above baseline, about 15% above baseline, or about 20% above baseline for one or more of the spirometric parameters compared to the same spirometric parameter obtained under a reference condition.

An inhalation solution that provides an onset of therapeutic effect of less than about 30 minutes, less than about 25 minutes, less than about 20 minutes, less than about 15 minutes, or less than about 10 minutes, in some embodiments, refers to an amount of time for the spirometric parameter to reach a predetermined threshold of about 5% above baseline, about 10% above baseline, about 15% above baseline, or about 20% above baseline.

In some embodiments, the methods or systems are provided for the treatment of acute exacerbations of Chronic Obstructive Pulmonary Disease (COPD), chronic bronchitis, and optionally emphysema in a patient, comprising administering to the patient a nominal dose of a LABA or a muscarinic antagonist in combination with a LABA in an aqueous inhalation solution at a concentration of a LABA or a muscarinic antagonist in combination with a LABA sufficient to provide a rapid onset of therapeutic effect and a long duration of therapeutic effect. In some embodiments, the rapid onset of therapeutic effect is less than about 30 minutes, less than about 25 minutes, less than about 20 minutes, less than about 15 minutes or less than about 10 minutes. In some embodiments, the long duration of therapeutic effect is at least about 12 hr to about 24 hr, about 18 hr to about 24 hr, about 20 hr to about 24 hr or at least about 18, 20, 24, 28, 30, 32 or 36 hr.

A time to maximum therapeutic effect means the amount of time for a preselected spirometric parameter to reach its peak level. In some embodiments, an enhanced time to maximum therapeutic effect means that administration of a LABA or a muscarinic antagonist in combination with a LABA with a high efficiency nebulizer, at a high concentration or both, results in a faster time to maximum therapeutic effect than would a dose of the LABA or the muscarinic antagonist in combination with a LABA administered with a conventional nebulizer. The parameters used to determine an enhanced time to maximum therapeutic effect may be one or more of FEV₁, FEV₁/FVC, PEF, FEF₂₅₋₇₅, FEF₂₅₋₅₀, FIF₂₅₋₇₅, or FIF₂₅₋₅₀.

Reduction in Adverse Side Effects

Conventional COPD therapy employing a LABA or a muscarinic antagonist with conventional inhalation devices and conventional nebulizers often results in deposition of pharmaceutically active ingredient in sections distinct from the pulmonary lung, e.g., mouth, throat, stomach, and optionally a esophagus. This is a result of the presence of muscarinic receptors on peripheral systems other than the pulmonary lung, for example in salivary glands, stomach, and elsewhere. Therefore the use of systemically active muscarinic antagonists is limited by side-effects such as, but not limited to, xerostomia (dry mouth), urinary hesitancy and retention, blurred vision, tachycardia, dizziness, insomnia, impotence, mental confusion and optionally a excitement, headache, anxiety, hypotension or palpitations.

In the present invention, the bronchodilation and other beneficial actions of a muscarinic antagonist in combination with a LABA are produced by an inhaled agent providing for a high therapeutic index for activity in the lung, i.e. lung deposition, compared with deposition of muscarinic antagonist in non-pulmonary regions, i.e. periphery compartments, mouth and pharynx. The present invention further provides for an inhalable muscarinic antagonist with low bioavailability in areas within a patient other than the lung (e.g. systemic bioavailability, local oropharyngeal or gastric regions), resulting in a decreased incidence and/or severity of systemic and/or local toxicity and/or side effects. A practitioner of ordinary skill can quantify a reduction in adverse side effects by measuring the incidence and/or severity of systemic and/or local toxicity and/or side effects in a given patient or patient population.

A reduced, or decreased, incidence or severity of systemic and/or local toxicity and/or side effects means that the method or system (e.g. a LABA or a muscarinic antagonist in combination with a LABA, administered with a conventional or high efficiency nebulizer) provides a decreased incidence and/or severity of systemic and/or local toxicity and/or side effects (for example dry mouth) in a given patient or patient population compared to a given reference therapy. In some embodiments, the reference therapy is administration of a LABA optionally in combination with a muscarinic antagonist with a conventional nebulizer. Some embodiments provide a method for the treatment or prophylaxis of a respiratory condition in a patient, comprising administering to the patient a nominal dose of a LABA or of a combination a muscarinic antagonist and LABA which, when administered with a high efficiency nebulizer, provides a calculated respirable dose of a LABA or a combination of a muscarinic antagonist and a LABA with a high efficiency nebulizer, wherein the calculated respirable dose of the LABA or combination of a muscarinic antagonist and a LABA administered with the high efficiency nebulizer demonstrates a decreased incidence and/or severity of systemic and/or local toxicity and/or side effects in the patient as compared to substantially the same calculated respirable dose of the LABA or combination of a muscarinic antagonist and a LABA administered with a conventional nebulizer. Some embodiments provide a method for the treatment or prophylaxis of a respiratory condition in a patient, comprising administering to the patient a nominal dose of said LABA or said combination of muscarinic antagonist and LABA which, when administered with a high efficiency nebulizer, provides a measured deposited dose of said LABA or said combination of a muscarinic antagonist and a LABA with a high efficiency nebulizer, wherein the measured deposited dose of a LABA or a combination of a muscarinic antagonist and a LABA administered with the high efficiency nebulizer demonstrates a decreased incidence and/or severity of systemic and/or local toxicity and/or side effects in the patient as compared to substantially the same measured deposited dose of a LABA or a combination of a muscarinic antagonist and a LABA administered with a conventional nebulizer. Some embodiments provide a system for performing the foregoing methods.

In some embodiments, administration of a LABA with a high efficiency nebulizer or coadministration of muscarinic antagonist and LABA (with or without a high efficiency nebulizer) reduces one or more side effects associated with the LABA, such as anxiety, hand tremors, muscle tremors, nervousness, dizziness, headache, hypokalemia, hyperglycemia, drowsiness, dyspnea, wheezing, drying or irritation of the oropharynx, coughing, chest pain, chest discomfort, palpitations, increased heart rate, tachycardia, bradycardia, angina, vertigo, central stimulation, insomnia, airway hyperreactivity (hypersensitivity), nausea, diarrhea, dry mouth, vomiting, anorexia, weakness, fatigue, flushed feeling, sweating, unusual taste, hoarseness, muscle cramps, or backaches.

In some embodiments, the method or system (e.g. LABA, with a high efficiency nebulizer or administration of a muscarinic antagonist in combination with a LABA, with a conventional or high efficiency nebulizer) provides for administering a muscarinic antagonist at a concentration of at least about 0.25 to about 2.0 mg/mL, at least about 0.25 mg/mL, at least about 0.5 mg/mL, at least about 1.0 mg/mL, at least about 1.5 mg/mL, or at least about 2.0 mg/mL and the muscarinic antagonist demonstrates a decreased incidence and optionally a severity of incidence and/or severity of systemic and/or local toxicity and/or side effects (for example dry mouth) in the patient as compared to substantially the same nominal dose of the muscarinic antagonist administered at a substantially lower concentration. In other embodiments, the concentration of muscarinic antagonist is about 0.05 to about 2.0 mg/mL, about 0.1 to 2.0 mg/mL, about 0.2 to about 2.0 mg/mL, about 0.05 to about 1.0 mg/mL, about 0.1 to about 1.0 mg/mL or about 0.2 to about 1.0 mg/mL. In some embodiments, the method or system (e.g. administration of a muscarinic antagonist in combination with a LABA, with a high efficiency nebulizer and/or at a high concentration) provides a method and/or inhalation system for administration of a muscarinic antagonist in a volume of about 0.5 mL or less, 1 mL or less, 1.5 mL or less, or 2.0 mL or less and wherein the muscarinic antagonist demonstrates less incidence and/or severity of systemic and/or local toxicity and/or side effects (for example dry mouth) in the patient as compared to substantially the same nominal dose of the muscarinic antagonist administered in a substantially higher volume of solution.

In some embodiments, the method or system (e.g., a combination of muscarinic antagonist with a LABA, with a conventional or high efficiency nebulizer) provides for methods and inhalation systems for reducing at least one side effect of the LABA and/or of the muscarinic antagonist and providing a duration of therapeutic effect of at least about 12 hr, about 12 hr to about 24 hr, about 18 hr to about 24 hr, about 20 hr to about 24 hr, or at least about 12, 18, 24, 28, 30, 32 or 36 hours. In some embodiments, the method or system (e.g., administration of a LABA or a muscarinic antagonist in combination with a LABA, with a high efficiency nebulizer and/or at a high concentration) provides for co-administration of other drugs and optionally excipients, for example an organic acid, such as ascorbic acid, citric acid or a mixture of both, pilocarpine, cevimeline or carboxymethylcellulose, or a mucolytic compound.

Enhanced Lung Deposition

Muscarinic receptors and beta 2-adrenoreceptors are widely distributed throughout the body. The ability to apply these active pharmaceutical agents (APIs) locally to the respiratory tract with sufficient lung deposition is particularly advantageous, as it would allow for administration of lower doses of the drug fostering increased patient compliance

The principle advantage of administration of a nebulized LABA or combination of muscarinic antagonist and LABA solution with a high efficiency nebulizer over other methods of pulmonary delivery of APIs is that such administration offers more efficient delivery of higher doses of said combination compared to conventional inhalation methods and systems, resulting in greater efficacy and a reduced incidence and/or a severity of side effects in the patient. In some embodiments, this allows for use of a higher nominal dose of API, as more efficient delivery of API to the lung is expected to result in lower proportional deposition in the mouth and pharynx, leading to reduced side effects from extrapulmonary (e.g. gastrointestinal) absorption of the API. In other embodiments, more efficient pulmonary delivery of API with a high efficiency nebulizer can permit use of a reduced nominal dose, relative to a nominal dose that is effective when administered with a conventional nebulizer, as more efficient lung delivery of the API means that more of the nominal dose reaches the target tissue and gives rise to the desired therapeutic effect. A more efficient delivery of said LABA or said combination is evidenced by direct delivery and deposition of the combination to the site of action, i.e. the lung (as used herein, “lung” refers to either or both the right and left lung organs). It can be assumed that substantially all of the combination delivered at the receptor site in the lungs will be absorbed into the blood plasma of the patient.

A lung deposition of 30% means 30% of the active ingredient in the inhalation device just prior to administration is deposited in the lung. A lung deposition of 60% means 60% of the active ingredient in the inhalation device just prior to administration is deposited in the lung, and so forth. Lung deposition can be determined using methods of scintigraphy or deconvolution of pharmacokinetic data. In some embodiments, the present invention provides for methods and inhalation systems for the treatment or prophylaxis of a respiratory condition in a patient, comprising administering to the patient a nominal dose of a LABA solution or a muscarinic antagonist in combination with a LABA with a high efficiency nebulizer inhalation device wherein administration of the muscarinic antagonist in combination with the LABA with the inhalation device provides lung deposition of the muscarinic antagonist in combination with a LABA of at least about 30%, at least about 35%, at least about 40%, at least about 45%, at least about 50%, at least about 55%, at least about 60%, about 30% to about 60%, about 30% to about 55%, about 30% to about 50%, about 30% to about 40%, about 30% to about 90%, about 40% to about 80%, about 50% to about 60%, or about 60% to about 70% based on the nominal dose of the LABA or of the muscarinic antagonist in combination with the LABA. In some embodiments, the present invention provides for methods and inhalation systems for the treatment or prophylaxis of a respiratory condition in a patient, comprising administering to the patient a nominal dose of a LABA or of a muscarinic antagonist in combination with the LABA in an aqueous inhalation solution with an inhalation device wherein administration of the LABA or the muscarinic antagonist in combination with a LABA with the inhalation device provides lung deposition of the LABA or the muscarinic antagonist and the LABA of at least about 15%, at least about 20%, at least about 25%, at least about 30%, at least about 35%, at least about 40%, at least about 45%, at least about 50%, at least about 55%, at least about 60%, about 20% to about 40%, about 25% to about 35%, about 25% to about 30%, about 35% to about 90%, about 40% to about 80%, about 50% to about 60%, or about 60% to about 70% based on the nominal dose of the LABA or the muscarinic antagonist and the LABA.

Aerosol particle/droplet size is one of the most important factors determining the deposition of aerosol drugs in the airways. The portion of an aerosol that has the highest probability of bypassing the upper airway and depositing in the lung measures between 1 and 5 μm. Particles larger than this are generally deposited in the oropharyngeal region and are swallowed, while sub-micron particles do not carry much drug and may be exhaled before deposition takes place. Smaller particles tend to deposit more peripherally in the lung than coarser particles, which may lead to a different clinical response. Consequently, differences in particle size of the aerosol emitted from inhalation devices may account for some of the variability in therapeutic efficacy and safety. Measurement of particle size, therefore, has an important role in guiding product development and in quality control of the marketed product.

The distribution of aerosol particle/droplet size can be expressed in terms of either or both of:

The Mass Median Aerodynamic Diameter (MMAD) and the Geometric Standard Deviation (GSD), wherein the MMAD is the droplet size at which half of the mass of the aerosol is contained in smaller droplets and half in larger droplets and the GSD is the geometric standard deviation of the particle population

The Fine Particle Fraction (FPF), which is the fraction of particles (which may be expressed as a percentage) that are <5 μm in diameter.

These measures have been used for comparisons of the in vitro performance of different inhaler device and drug combinations. In general, the higher the fine particle fraction, the higher the proportion of the emitted dose that is likely to reach the lung.

There are two main methods used to measure aerosol deposition in the lungs. First, γ-scintigraphy is performed by radiolabeling the drug with a substance like 99m-technetium, and scanning the subject after inhalation of the drug. This technique has the advantage of being able to quantify the proportion of aerosol inhaled by the patient, as well as regional distribution in the upper airway and lungs. Second, since most of the drug that is deposited in the lower airways will be absorbed into the bloodstream, pharmacokinetic techniques are used to measure lung deposition. This technique can assess the total amount of drug that interacts with the airway epithelium and is absorbed systemically, but will miss the small portion that may be expectorated or swallowed after mucociliary clearance, and does not fully describe regional distribution. Therefore, γ-scintigraphy and pharmacokinetic studies are in many cases considered complementary.

The relationship between pulmonary deposition of inhaled beta 2-agonists and therapeutic effect is now well-established, since the immediate effects of these agents on the airways are relatively easy to measure. As the pulmonary dose-response curve for the beta 2-agonists is sigmoidal (i.e. an initial slope followed by a plateau), increasing the dose deposited in the lung will elicit an increased therapeutic effect only if the initial dose was on the rising slope of the dose-response curve.

Lung deposition of a particular drug is influenced by the mass of fluid contained in the nebulized droplets administered to a patient with a particular Mass Median Aerodynamic Diameter (MMAD) and Geometric Standard Deviation (GSD). In general, there is an inverse relationship between the average MMAD and GSD of a particular nebulizer's emitted droplets and deposition of the droplets in a patient's lung. Therefore, a smaller MMAD results in an increased likelihood of lung deposition in a patient. Likewise, when the MMAD is in the range of about 4-5 μm, a narrower GSD results in a higher degree of lung deposition, since a higher percentage of particles will be under 5 μm in diameter. It is believed that, in general, aerosol particles greater than ˜10 μm in aerodynamic diameter deposit primarily in the oropharynx and are swallowed rather than reaching the lungs. Because of the plausible link between MMAD and GSD values and eventual deposition site within the respiratory tract, smaller MMAD and GSD values may affect both the safety (by reducing non-pulmonary deposition and possibly thereby reducing local and potentially systemic effects) and the efficacy (by increasing the amount of drug actually deposited in the lungs) of drug products administered with such high efficiency inhalation devices. Laser-diffraction provides for an in-vitro method of determining MMAD and GSD data, which can then be plotted onto what usually results in a log-normal shaped curve (depicting mass distribution % on the Y-axis and droplet diameter on the X-axis). Laser-diffraction methods are well-known to one of ordinary skill in the art. In addition to laser-diffraction methods, in-vitro data for MMAD and GSD can also be measured using cascade impaction or time-of-flight analytical methods, both of which are known to one of ordinary skill in the art.

In some embodiments, administration of the LABA or the combination of muscarinic antagonist and LABA with the high efficiency nebulizer provides a Geometric Standard Deviation (GSD) of emitted droplet size distribution of the solution administered with a high efficiency nebulizer of about 1.1 to about 2.1, about 1.2 to about 2.0, about 1.3 to about 1.9, about 2.2, at least about 1.4 to about 1.8, at least about 1.5 to about 1.7, about 1.4, about 1.5, or about 1.6. In some embodiments, administration of API with a high efficiency nebulizer provides a Mass Median Aerodynamic Diameter (MMAD) of droplet size of the solution emitted with the high efficiency nebulizer of about 1 μm to about 5 μm, about 2 to about 4 μm, or about 3.5 to about 4.5 μm.

Respirable Fraction (RF), Emitted Dose (ED), Respirable Dose (RD) and the Respirable Dose Delivery Rate (RDDR) provide technical dimensions for the efficiency of a nebulizer inhalation device. RF is a generally accepted estimate of lung deposition within the medical community. RF represents the fraction of the delivered aerosol dose, or inhaled mass, with droplets of diameter less than 5.0 μm. Droplets of less than 5.0 μm in diameter are considered to penetrate to the lung. In some embodiments, administration of the LABA or muscarinic antagonist (e.g. LAMA) in combination with a LABA with an aqueous inhalation device provides a respirable fraction (RF) of API of at least about 60%, at least about 65%, at least about 70%, at least about 75%, at least about 80%, at least about 85%, at least about 90%, about 60% to about 95%, about 65% to about 95%, or about 70% to about 90%.

The Emitted Dose (ED) portion of drug that is actually emitted from the mouthpiece of the device. The ED of the muscarinic antagonist in combination with a LABA is to be tested under simulated breathing conditions using a standardized bench setup, which are known to one of skill in the art. In some embodiments, the ED of the LABA or combination of muscarinic antagonist and LABA is at least about 30%, at least about 35%, at least about 40%, at least about 45%, at least about 50%, at least about 55%, at least about 60%, about 30% to about 60%, about 30% to about 55%, about 30% to about 50%, about 30% to about 40%, about 30% to about 75%, about 40% to about 70%, or about 45% to about 60%.

### EXAMPLES

The following non-limiting examples provide ingredients, processes and procedures for practicing the systems and methods herein, and are intended to be illustrative of the invention described and claimed herein. The procedures below describe some embodiments of methods of delivery of a nebulized long-acting beta 2-agonist (LABA) with a high efficiency or a muscarinic antagonist in combination with a nebulized beta 2-agonist aqueous solution (in combination therapy) with a high efficiency nebulizer, as described herein.

### Example 1

### Randomized, Cross-Over, Single Dose Study

Approximately twelve (12) adult COPD patients of ages 40-75 years are randomized to receive five treatments in a crossover design: (1) 20 μg formoterol administered with a conventional nebulizer; (2) 5 μg of formoterol administered with a high efficiency nebulizer; (3) 7.5 μg of formoterol administered with a high efficiency nebulizer; (4) 10 μg of formoterol administered with a high efficiency nebulizer: and (5) 20 μg of formoterol administered with a high efficiency nebulizer.

Lung function is determined by spirometry, which measures e.g. FEV₁ and optionally other suitable spirometry parameters, such as FEV₁ AUC. Spirometry is conducted immediately before and at predetermined intervals following administration of the formoterol to the patients. Additionally, the patients are monitored for any adverse events, such as tremor, as well as for vital signs and electrocardiogram. COPD symptom scores are obtained by administering to each patient a conventional or proprietary symptom score instrument.

A projected outcome is that formoterol administered to patients with a high efficiency nebulizer at the tested doses produces in a patient or population of patients a therapeutic effect (i.e. at least one spirometry measurement, e.g. FEV₁ is at least 10% and/or 100 mL above baseline and/or placebo for a significant period of time, e.g. 12-24 hours.)

Another projected outcome is that formoterol produces clinically meaningful bronchodilation of at least 24 hours when administered with a high efficiency nebulizer, wherein the same or higher dose of formoterol produces less than 24 hours of clinically meaningful bronchodilation when administered with a conventional nebulizer.

Another projected outcome is that a lower dose formoterol administered to patients with a high efficiency nebulizer produces in a patient or population of patients improved or similar therapeutic effects with an improved adverse event profile and/or improved side effects as a measure of cellular activity (changes in serum potassium, glucose levels) as compared to a selected dose of formoterol administered with a conventional nebulizer.

### Example 2

### Randomized, Double-Blind, Placebo-Controlled, Parallel-Group, Multi-Dose Study

Approx 50 adult COPD patients of ages 40-75 years are randomized to one of five treatment groups: (1) 20 μg formoterol administered B.I.D. with a conventional nebulizer; (2) 10 μg of formoterol administered B.I.D. with a high efficiency nebulizer; (3) 10 μg of formoterol administered Q.D. with a high efficiency nebulizer; (4) 5 μg of formoterol administered Q.D. with a high efficiency nebulizer; (5) placebo administered B.I.D. with a high efficiency nebulizer.

Lung function is determined by spirometry, which measures e.g. FEV₁ and optionally other suitable spirometry parameters, such as FEV₁ AUC. Spirometry is conducted immediately before and at predetermined intervals following administration of the formoterol to the patients. Additionally, the patients are monitored for any adverse events, such as tremor, as well as for vital signs and electrocardiogram. COPD symptom scores are obtained by administering to each patient a conventional or proprietary symptom score instrument.

A projected outcome is that formoterol administered to patients with a high efficiency nebulizer at the tested doses produces in a patient or population of patients a therapeutic effect (i.e. at least one spirometry measurement, e.g. FEV₁ is at least 10% and/or 100 mL above baseline and/or placebo for a significant period of time, e.g. 12-24 hours.)

Another projected outcome is that formoterol produces clinically meaningful bronchodilation of at least 24 hours when administered with a high efficiency nebulizer, wherein the same or higher dose of formoterol produces less than 24 hours of clinically meaningful bronchodilation when administered with a conventional nebulizer.

Another projected outcome is that lower dose formoterol administered to patients with a high efficiency nebulizer produces in a patient or population of patients improved or similar therapeutic effects with an improved adverse event profile and/or improved side effects as a measure of cellular activity (changes in serum potassium, glucose levels) as compared to a selected dose of formoterol administered with a conventional nebulizer.

### Example 3

### Randomized, Double-Blind, Placebo-Controlled Cross-Over, Single Dose Study

Approx twelve (12) adult COPD patients of ages 40-75 years are randomized to receive five treatments in a cross-over design: (1) 15 μg arformoterol administered with a conventional nebulizer; (2) 8 μg of arformoterol administered with a high efficiency nebulizer; (3) 4 μg of arformoterol administered with a high efficiency nebulizer; (4) 2 μg of arformoterol administered with a high efficiency nebulizer and (5) nebulized placebo.

Lung function is determined by spirometry, which measures e.g. FEV₁ and optionally other suitable spirometry parameters, such as FEV₁ AUC. Spirometry is conducted immediately before and at predetermined intervals following administration of the arformoterol to the patients. Additionally, the patients are monitored for any adverse events, such as tremor, as well as for vital signs and electrocardiogram. COPD symptom scores are obtained by administering to each patient a conventional or proprietary symptom score instrument.

A projected outcome is that arformoterol administered to patients with a high efficiency nebulizer at the tested doses produces in a patient or population of patients a therapeutic effect (i.e. at least one spirometry measurement, e.g. FEV₁ is at least 10% and/or 100 mL above baseline and/or placebo for a significant period of time, e.g. 12-24 hours.)

Another projected outcome is that arformoterol produces clinically meaningful bronchodilation of at least 24 hours when administered with a high efficiency nebulizer, wherein the same or higher dose of arformoterol produces less than 24 hours of clinically meaningful bronchodilation when administered with a conventional nebulizer.

Another projected outcome is that lower dose arformoterol administered to patients with a high efficiency nebulizer produces in a patient or population of patients improved or similar therapeutic effects with an improved adverse event profile and/or improved side effects as a measure of cellular activity (changes in serum potassium, glucose levels) as compared to a selected dose of arformoterol administered with a conventional nebulizer.

### Example 4

### Randomized, Double-Blind, Placebo-Controlled, Parallel-Group, Multi-Dose Study

Approx fifty (50) adult COPD patients of ages 40-75 years are randomized to one of five treatment groups: (1) 15 μg arformoterol administered B.I.D. with a conventional nebulizer; (2) 8 μg of arformoterol administered B.I.D. with a high efficiency nebulizer; (3) 8 μg of arformoterol administered Q.D. with a high efficiency nebulizer; (4) 4 μg of arformoterol administered B.I.D. with a high efficiency nebulizer; and (5) placebo administered B.I.D. with a high efficiency nebulizer.

Lung function is determined by spirometry, which measures e.g. FEV₁ and optionally other suitable spirometry parameters, such as FEV₁ AUC. Spirometry is conducted immediately before and at predetermined intervals following administration of the arformoterol to the patients. Additionally, the patients are monitored for any adverse events, such as tremor, as well as for vital signs and electrocardiogram. COPD symptom scores are obtained by administering to each patient a conventional or proprietary symptom score instrument.

A projected outcome is that arformoterol administered to patients with a high efficiency nebulizer at the tested doses produces in a patient or population of patients a therapeutic effect (i.e. at least one spirometry measurement, e.g. FEV₁ is at least 10% and/or 100 mL above baseline and/or placebo for a significant period of time, e.g. 12-24 hours.)

Another projected outcome is that arformoterol produces clinically meaningful bronchodilation of at least 24 hours when administered with a high efficiency nebulizer, wherein the same or higher dose of arformoterol produces less than 24 hours of clinically meaningful bronchodilation when administered with a conventional nebulizer.

Another projected outcome is that lower dose arformoterol administered to patients with a high efficiency nebulizer produces in a patient or population of patients improved or similar therapeutic effects with an improved adverse event profile and/or improved side effects as a measure of cellular activity (changes in serum potassium, glucose levels) as compared to a selected dose of arformoterol administered with a conventional nebulizer.

### Example 5

### Randomized, Placebo-Controlled, Parallel-Group, Multi-Dose Study

At least about three hundred (300) adult human COPD patients of ages >45 years are randomized to one of three treatment groups: (1) formoterol or arformoterol administered with a high efficiency nebulizer; (2) formoterol or arformoterol administered with a conventional nebulizer; (3) placebo.

Lung function is determined by spirometry, which measures e.g. FEV₁ and optionally other suitable spirometry parameters, such as FEV₁ AUC. Spirometry is conducted immediately before and at predetermined intervals following administration of the arformoterol to the patients. Additionally, the patients are monitored for any adverse events, such as tremor, as well as for vital signs and electrocardiogram. COPD symptom scores are obtained by administering to each patient a conventional or proprietary symptom score instrument.

A projected outcome is that formoterol or arformoterol administered to patients with a high efficiency nebulizer at the tested doses produces in a patient or population of patients a therapeutic effect (i.e. at least one spirometry measurement, e.g. FEV₁ is at least 10% and/or 100 mL above baseline and/or placebo for a significant period of time, e.g. 12-24 hours.)

Another projected outcome is that formoterol or arformoterol produces clinically meaningful bronchodilation of at least 24 hours when administered with a high efficiency nebulizer, wherein the same or higher dose of formoterol or arformoterol produces less than 24 hours of clinically meaningful bronchodilation when administered with a conventional nebulizer.

Another projected outcome is that a lower dose formoterol or arformoterol administered to patients with a high efficiency nebulizer produces in a patient or population of patients improved or similar therapeutic effects with an improved adverse event profile and/or improved side effects as a measure of cellular activity (changes in serum potassium, glucose levels) as compared to a selected dose of formoterol or arformoterol administered with a conventional nebulizer.

### Example 6

### Randomized, Cross-Over, Single Dose Study

At least about eight (8) adult healthy human volunteers (patients) are randomized to receive four treatments in a cross-over design: (1) 50 μg of salmeterol; (3) 25 μg of salmeterol administered with a high efficiency nebulizer; (4) 12 μg of salmeterol administered with a high efficiency nebulizer. Lung function is determined by spirometry, which measures e.g. FEV₁ and optionally other suitable spirometry parameters, such as FEV₁ AUC.

Lung function is determined by spirometry, which measures e.g. FEV₁ and optionally other suitable spirometry parameters, such as FEV₁ AUC. Spirometry is conducted immediately before and at predetermined intervals following administration of the salmeterol to the patients. Additionally, the patients are monitored for any adverse events, such as tremor, as well as for vital signs and electrocardiogram. COPD symptom scores are obtained by administering to each patient a conventional or proprietary symptom score instrument.

A projected outcome is that salmeterol administered to patients with a high efficiency nebulizer at the tested doses produces in a patient or population of patients a therapeutic effect (i.e. at least one spirometry measurement, e.g. FEV₁ is at least 10% and/or 100 mL above baseline and/or placebo for a significant period of time, e.g. 12-24 hours.)

Another projected outcome is that salmeterol produces clinically meaningful bronchodilation of at least 24 hours when administered with a high efficiency nebulizer, wherein the same dose of salmeterol produces less than 24 hours of clinically meaningful bronchodilation when administered with a conventional nebulizer, metered dose inhaler, or dry powder inhaler.

Another projected outcome is that lower dose salmeterol administered to patients with a high efficiency nebulizer produces in a patient or population of patients improved or similar therapeutic effects with an improved adverse event profile and/or improved side effects as a measure of cellular activity (changes in serum potassium, glucose levels) as compared to a selected dose of salmeterol administered with a conventional nebulizer.

### Example 7

### Randomized, Cross-Over, Single Dose Study (Glycopyrrolate+Formoterol (Racemate))

Approx. 36 adult COPD patients of ages 40-75 years are randomized to receive single dose treatments in a crossover design using a high efficiency nebulizer: (1) a first dose of glycopyrrolate (e.g. a dose in the range of 100-300 meg); (2) a first dose of formoterol (racemate) (e.g. a dose in the range of 5-20 meg); (3) the first dose of glycopyrrolate from (1) and the first dose of formoterol (racemate) from (2); (4) the first dose of glycopyrrolate from (1) and a second dose of formoterol (racemate), which is approximately half the formoterol dose in (2); (5) a second dose of glycopyrrolate, which is approximately half the first glycopyrrolate dose from (1), and the first dose of formoterol (racemate) from (2); (6) the second dose of glycopyrrolate (approximately half the first dose from (1)) and the second dose of formoterol (racemate) (approximately half the dose in (2)); (7) a third dose of glycopyrrolate, which is approximately one quarter the dose in (1), and the first dose of formoterol from (2); (8) the third dose of glycopyrrolate (approximately one quarter of the dose in (1)), and the second dose of formoterol (approximately half the dose in (2)); (9) Placebo.

Blood and/or urine samples are drawn immediately prior to administration of glycopyrrolate and formoterol and at predetermined time points thereafter. The blood plasma levels of glycopyrrolate in the blood samples and urine levels of formoterol in the urine are determined and analyzed to determine the appropriate pharmacokinetic parameters (e.g. C., Tₘₐₓ, AUCₗₐₛₜ, and AUC_{0-∞}) for glycopyrrolate. Additionally, the patients are monitored for any adverse events as well as vital signs and electrocardiogram.

Lung function is determined by spirometry, which measures e.g. FEV₁ and optionally other suitable spirometry parameters, such as FEV₁ AUC. Spirometry is conducted immediately before and at predetermined intervals following administration of the arformoterol to the patients.

A projected outcome is that administration of a standard dose of combination of formoterol and glycopyrrolate with a high efficiency nebulizer will result in significantly improved therapeutic effect compared to administration of formoterol with a nebulizer as a monotherapy and/or compared to administration of glycopyrrolate with a nebulizer as a monotherapy. Another projected outcome is that combined glycopyrrolate and formoterol therapy results in at least 24 hours of clinically meaningful bronchodilation with acceptable side effects. Another projected outcome is that glycopyrrolate and formoterol therapy results in reduced side effects as compared to dosing of either of the therapeutic agents separately. A further projected outcome is that combined dosing of a glycopyrrolate and formoterol permits dosing at less than half a standard dose of one or both of the glycopyrrolate and/or formoterol.

### Example 8

### Randomized, Cross-Over, Single Dose Study (Glycopyrrolate+Arformoterol)

Approx 15 adult healthy human volunteers (patients) are randomized to receive treatments in a cross-over design to be administered, with a high efficiency nebulizer: (1) 200 mcg glycopyrrolate administered; (2) 8 μg of arformoterol (R,R-formoterol, at least 90% enantiomerically pure); (3) 200 mcg of glycopyrrolate and 8 μg of arformoterol; (4) 200 mcg of glycopyrrolate and 4 μg of arformoterol; (5) 100 mcg of glycopyrrolate and 8 μg of arformoterol; (6) 100 mcg of glycopyrrolate and 4 μg of arformoterol; (7) 50 mcg of glycopyrrolate and 8 μg of arformoterol; (8) 50 mcg of glycopyrrolate and 4 μg of arformoterol; (9) Placebo.

Blood and/or urine samples are drawn immediately prior to administration of glycopyrrolate and arformoterol and at predetermined time points thereafter. The blood plasma levels of glycopyrrolate in the blood samples and urine levels of arformoterol in the urine are determined and analyzed to determine the appropriate pharmacokinetic parameters (e.g. C., Tₘₐₓ, AUCₗₐₛₜ, and AUC_{O-∞}) for glycopyrrolate. Additionally, the patients are monitored for any adverse events as well as vital signs and electrocardiogram.

Lung function is determined by spirometry, which measures e.g. FEV₁ and optionally other suitable spirometry parameters, such as FEV₁ AUC. Spirometry is conducted immediately before and at predetermined intervals following administration of the combination of glycopyrrolate and arformoterol to the patients.

A projected outcome is that administration of a standard (approved) dose of arformoterol with a high efficiency nebulizer will result in a therapeutic effect for at least 24 hr. Another projected outcome is that administration of a standard dose of combination of arformoterol and glycopyrrolate with a high efficiency nebulizer will result in significantly improved therapeutic effect compared to administration of arformoterol with a nebulizer as a monotherapy and/or compared to administration of glycopyrrolate with a nebulizer as a monotherapy. Another projected outcome is that combined glycopyrrolate and arformoterol therapy permits 24 hour dosing. Another projected outcome is that combined glycopyrrolate and arformoterol therapy results in reduced side effects as compared to dosing of either of the therapeutic agents separately. A further projected outcome is that combined dosing of glycopyrrolate and arformoterol permits dosing at less than half a standard dose of one or both of the glycopyrrolate and/or the arformoterol.

Another projected outcome is that arformoterol administered to human patients with a high efficiency nebulizer at a lower dose produces in a patient or population of patients a pharmacokinetic profile characterized by a Cₘₐₓ, AUCₗₐₛₜ and/or AUC_{O-∞} that is comparable to, or greater than, a Cₘₐₓ, AUCₗₐₛₜ and/or AUC_{O-∞} obtained with a higher dose of arformoterol administered with a conventional nebulizer.

Another projected outcome is that arformoterol administered to human patients with a high efficiency nebulizer produces in a patient or population of patients an improved adverse event profile as compared to a comparable or lower dose of arformoterol administered with a conventional nebulizer.

Another projected outcome is that arformoterol administered to human patients with a high efficiency nebulizer produces in a patient or population of patients higher degree of lung deposition of the arformoterol as compared to a comparable or higher dose of arformoterol administered with a conventional nebulizer.

### Example 9

### Randomized, Double-Blind, Placebo-Controlled, Multi-Dose Study

Approx. twenty-four (24) adult COPD patients of ages 40-75 years are randomized to receive five treatments administered with a high efficiency nebulizer in a cross-over designs: (1) 100 mcg glycopyrrolate Q.D.; (2) 10 mcg of formoterol administered B.I.D.; (3) 100 mcg of glycopyrrolate Q.D. and 10 mcg of formoterol administered B.I.D.; (4) 100 mcg of glycopyrrolate Q.D. and 10 mcg of formoterol Q.D.; and (5) placebo.

Lung function is determined by spirometry, which measures e.g. FEV₁ and optionally other suitable spirometry parameters, such as FEV₁ AUC. Spirometry is conducted immediately before and at predetermined intervals following administration of the arformoterol to the patients. Additionally, the patients are monitored for any adverse events, as well as for vital signs and electrocardiogram.

Lung function is determined by spirometry, which measures e.g. FEV₁ and optionally other suitable spirometry parameters, such as FEV₁ AUC. Spirometry is conducted immediately before and at predetermined intervals following administration of the formoterol to the patients. A projected outcome is that combined glycopyrrolate and formoterol therapy permits 24 hour dosing. Another projected outcome is that combined glycopyrrolate and formoterol therapy results in reduced side effects as compared to dosing of either of the therapeutic agents separately. A further projected outcome is that combined dosing of glycopyrrolate and formoterol permits dosing at less than half a standard dose of one or both of the glycopyrrolate and/or formoterol.

### Example 10

### Randomized, Double-blind, Placebo-Controlled, Parallel-Group, Multi-Dose Study

Approx. five hundred (500) adult COPD patients of ages 40-75 years are randomized to receive one of four treatments administered with a high efficiency nebulizer: (1) Glycopyrrolate Q.D; (2) Formoterol administered Q.D.; (3) Glycopyrrolate and formoterol administered Q.D.; (4) Placebo.

Lung function is determined by spirometry, which measures e.g. FEV₁ and optionally other suitable spirometry parameters, such as FEV₁ AUC. Spirometry is conducted immediately before and at predetermined intervals following administration of the salmeterol to the patients. A projected outcome is that combined glycopyrrolate and formoterol therapy permits 24 hour dosing. Another projected outcome is that combined glycopyrrolate and formoterol therapy results in reduced side effects as compared to dosing of either of the therapeutic agents separately. A further projected outcome is that combined dosing of a glycopyrrolate and formoterol permits dosing at less than half a standard dose of one or both of glycopyrrolate and/or formoterol.

While preferred embodiments of the present invention have been shown and described herein, it will be apparent that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a patient having chronic obstructive pulmonary disease (COPD), comprising administering to the patient with a high efficiency nebulizer a dose of formoterol, or a pharmaceutically acceptable salt thereof, and a dose of glycopyrrolate, or a pharmaceutically acceptable salt thereof.

2. The method of claim 1, wherein the administration with the high efficiency nebulizer produces an improved magnitude or duration of a therapeutic effect in the patient compared to administration of formoterol, or a pharmaceutically acceptable salt thereof, or glycopyrrolate, or a pharmaceutically acceptable salt thereof, with a conventional nebulizer, a metered dose inhaler, or a dry powder inhaler as a monotherapy.

3. The method of claim 2, wherein the improved magnitude or duration of a therapeutic effect comprises clinically meaningful bronchodilation for at least 24 hours.

4. The method of claim 3, wherein the clinically meaningful bronchodilation comprises an increase in trough FEV₁ of at least 10% or at least 100 mL above placebo.

5. The method of claim 1, wherein the administration with the high efficiency nebulizer of formoterol, or a pharmaceutically acceptable salt thereof, and glycopyrrolate, or a pharmaceutically acceptable salt thereof, produces improved or reduced side effects in the patient compared to administration of formoterol, or a pharmaceutically acceptable salt thereof, or glycopyrrolate, or a pharmaceutically acceptable salt thereof, with a conventional nebulizer, a metered dose inhaler, or a dry powder inhaler as a monotherapy.

6. The method of claim 1, wherein the dose of formoterol, or a pharmaceutically acceptable salt thereof, administered to the patient with the high efficiency nebulizer is less than half of an approved therapeutic dose of formoterol, or a pharmaceutically acceptable salt thereof, administered with a conventional nebulizer, a metered dose inhaler, or a dry powder inhaler.

7. The method of claim 1, wherein the dose of formoterol, or a pharmaceutically acceptable salt thereof, administered to the patient with the high efficiency nebulizer is about 0.5 μg to about 15 μg.

8. The method of claim 1, wherein the dose of formoterol, or a pharmaceutically acceptable salt thereof, administered to the patient with the high efficiency nebulizer is about 0.5 μg to about 2 μg.

9. The method of claim 1, wherein the dose of glycopyrrolate, or a pharmaceutically acceptable salt thereof, administered to the patient with the high efficiency nebulizer is less than half of an approved therapeutic dose of glycopyrrolate, or a pharmaceutically acceptable salt thereof, administered with a conventional nebulizer, a metered dose inhaler, or a dry powder inhaler.

10. The method of claim 1, wherein the dose of formoterol, or a pharmaceutically acceptable salt thereof, and the dose of glycopyrrolate, or a pharmaceutically acceptable salt thereof, is administered to the patient twice daily.

11. A method of treating a patient having chronic obstructive pulmonary disease (COPD), comprising administering to the patient with a high efficiency nebulizer a dose of R,R-formoterol, or a pharmaceutically acceptable salt thereof, and a dose of glycopyrrolate, or a pharmaceutically acceptable salt thereof.

12. The method of claim 11, wherein the administration with the high efficiency nebulizer produces an improved magnitude or duration of a therapeutic effect in the patient compared to administration of R,R-formoterol, or a pharmaceutically acceptable salt thereof, or glycopyrrolate, or a pharmaceutically acceptable salt thereof, with a conventional nebulizer, a metered dose inhaler, or a dry powder inhaler as a monotherapy.

13. The method of claim 12, wherein the improved magnitude or duration of a therapeutic effect comprises clinically meaningful bronchodilation for at least 24 hours.

14. The method of claim 13, wherein the clinically meaningful bronchodilation comprises an increase in trough FEV₁ of at least 10% or at least 100 mL above placebo.

15. The method of claim 11, wherein the administration with the high efficiency nebulizer of R,R-formoterol, or a pharmaceutically acceptable salt thereof, and glycopyrrolate, or a pharmaceutically acceptable salt thereof, produces improved or reduce d side effects in the patient compared to administration of R,R-formoterol, or a pharmaceutically acceptable salt thereof, or glycopyrrolate, or a pharmaceutically acceptable salt thereof, with a conventional nebulizer, a metered dose inhaler, or a dry powder inhaler as a monotherapy.

16. The method of claim 11, wherein the dose of R,R-formoterol, or a pharmaceutically acceptable salt thereof, administered to the patient with the high efficiency nebulizer is less than half of an approved therapeutic dose of R,R-formoterol, or a pharmaceutically acceptable salt thereof, administered with a conventional nebulizer, a metered dose inhaler, or a dry powder inhaler.

17. The method of claim 11, wherein the dose of R,R-formoterol, or a pharmaceutically acceptable salt thereof, administered to the patient with the high efficiency nebulizer is about 0.5 μg to about 15 μg.

18. The method of claim 11, wherein the dose of R,R-formoterol, or a pharmaceutically acceptable salt thereof, administered to the patient with the high efficiency nebulizer is about 0.5 μg to about 2 μg.

19. The method of claim 11, wherein the dose of glycopyrrolate, or a pharmaceutically acceptable salt thereof, administered to the patient with the high efficiency nebulizer is less than half of an approved therapeutic dose of glycopyrrolate, or a pharmaceutically acceptable salt thereof, administered with a conventional nebulizer, a metered dose inhaler, or a dry powder inhaler.

20. The method of claim 11, wherein the dose of R,R-formoterol, or a pharmaceutically acceptable salt thereof, and the dose of glycopyrrolate, or a pharmaceutically acceptable salt thereof, is administered to the patient twice daily.
